(19) United States
(12) Patent Application Publication
(10) Pub. No.: US 2007/0265345 A1
(43) Pub. Date: Nov. 15, 2007

(54) Novel Biphenyl Compounds And Their Use

(75) Inventors: Amita M. Chaudhari, Collegeville, PA (US); Dashyant Dhanak, Collegeville, PA (US); Steven David Knight, Collegeville, PA (US); David J. Morgans JR., King of Prussia, PA (US); Cynthia A. Parrish, College of Prussia, PA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignees: SMITHKLINE BEECHAM CORPORATION, Philadelphia, PA (US); CYTOKINETICS, South San Francisco, CA (US)

(21) Appl. No.: 11/632,331

(22) PCT Filed: Jul. 27, 2005
(86) PCT No.: PCT/US05/26214

§ 371 Date: Jan. 12, 2007

Related U.S. Application Data
(60) Provisional application No. 60/591,550, filed on Jul. 27, 2004.

(51) Int. Cl. A61K 31/17; A61K 31/165; A61P 35/00; A61P 37/00; A61P 43/00; A61P 9/00; C07C 233/00; C07C 273/18; C07C 275/00

(52) U.S. Cl. 514/595; 514/618; 564/161; 564/54

(57) ABSTRACT

The invention is directed to certain biphenyl compounds. Specifically, the invention is directed to compounds according to Formula I: 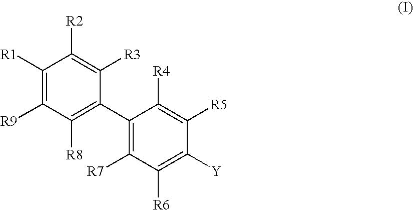 wherein R1, R2, R3, R4, R5, R6, R7, R8, R9, and Y are as defined below, and to pharmaceutically-acceptable salts thereof. The compounds of the invention are KSP inhibitors, particularly human KSP inhibitors, and can be useful for the treatment for a variety of diseases and conditions, such as cancer, hyperplasias, restenosis, cardiac hypertrophy, immune disorders, fungal disorders, and inflammation. Accordingly, the invention is further directed to pharmaceutical compositions comprising a compound of the invention. The invention is still further directed to methods of inhibiting KSP and treatment of conditions associated therewith using a compound of the invention or a pharmaceutical composition comprising a compound of the invention. In an additional aspect, the invention provides methods of screening for compounds that will bind to a KSP kinesin, for example compounds that will displace or compete with the binding of the compounds of the invention. The methods comprise combining a labeled compound of the invention, a KSP kinesin, and at least one candidate agent and determining the binding of the candidate bioactive agent to the KSP kinesin. In a further aspect, the invention provides methods of screening for modulators of KSP kinesin activity. The methods comprise combining a compound of the invention, a KSP kinesin, and at least one candidate agent and determining the effect of the candidate bioactive agent on the KSP kinesin activity.

### FIELD OF THE INVENTION

This invention is directed to certain biphenyl compounds which are inhibitors of the mitotic kinesin KSP and are useful in the treatment of cellular proliferative diseases.

### BACKGROUND OF THE INVENTION

Among therapeutic agents used to treat cancer are the taxanes and vinca alkaloids, which act on microtubules. Microtubules are the primary structural element of the mitotic spindle. The mitotic spindle is responsible for distribution of replicate copies of the genome to each of the two daughter cells that result from cell division. It is presumed that disruption of the mitotic spindle by these drugs results in inhibition of cancer cell division, and induction of cancer cell death. However, microtubules form other types of cellular structures, including tracks for intracellular transport in nerve processes. Because these agents do not specifically target mitotic spindles, they have side effects that limit their usefulness.

Improvements in the specificity of agents used to treat cancer is of considerable interest because of the therapeutic benefits which would be realized if the side effects associated with the administration of these agents could be reduced. Traditionally, dramatic improvements in the treatment of cancer are associated with identification of therapeutic agents acting through novel mechanisms. Examples of this include not only the taxanes, but also the camptothecin class of topoisomerase I inhibitors. From both of these perspectives, mitotic kinesins are attractive targets for new anti-cancer agents.

Mitotic kinesins are enzymes essential for assembly and function of the mitotic spindle, but are not generally part of other microtubule structures, such as in nerve processes. Mitotic kinesins play essential roles during all phases of mitosis. These enzymes are “molecular motors” that transform energy released by hydrolysis of ATP into mechanical force which drives the directional movement of cellular cargoes along microtubules. The catalytic domain sufficient for this task is a compact structure of approximately 340 amino acids. During mitosis, kinesins organize microtubules into the bipolar structure that is the mitotic spindle. Kinesins mediate movement of chromosomes along spindle microtubules, as well as structural changes in the mitotic spindle associated with specific phases of mitosis. Experimental perturbation of mitotic kinesin function causes malformation or dysfunction of the mitotic spindle, frequently resulting in cell cycle arrest and cell death.

Among the mitotic kinesins which have been identified is KSP. KSP belongs to an evolutionarily conserved kinesin subfamily of plus end-directed microtubule motors that assemble into bipolar homotetramers consisting of antiparallel homodimers. During mitosis KSP associates with microtubules of the mitotic spindle. Microinjection of antibodies directed against KSP into human cells prevents spindle pole separation during prometaphase, giving rise to monopolar spindles and causing mitotic arrest and induction of programmed cell death. KSP and related kinesins in other, non-human, organisms, bundle antiparallel microtubules and slide them relative to one another, thus forcing the two spindle poles apart. KSP may also mediate in anaphase B spindle elongation and focussing of microtubules at the spindle pole.

Human KSP (also termed HsEg5) has been described (Blangy, et al., Cell, 83:1159-69 (1995); Whitehead, et al., Arthritis Rheum., 39:1635-42 (1996); Galgio et al., J. Cell Biol., 135:339-414 (1996); Blangy, et al., J. Biol. Chem., 272:19418-24 (1997); Blangy, et al., Cell Motil Cytoskeleton, 40:174-82 (1998); Whitehead and Rattner, J. Cell Sci., 111:2551-61 (1998); Kaiser, et al., JBC 274:18925-31 (1999); GenBank accession numbers: X85137, NM004523 and U37426), and a fragment of the KSP gene (TRIP5) has been described (Lee, et al., Mol. Endocrinol., 9:243-54 (1995); GenBank accession number L40372). Xenopus KSP homologs (Eg5), as well as Drosophila KLP61 F/KRP130 have been reported.

In view of the role KSP plays in cell mitosis, compounds that inhibit KSP may be useful in the treatment of diseases of proliferating cells such as cancer, hyperplasias, restenosis, cardiac hypertrophy, immune disorders, fungal disorders, and inflammation. Thus there remains a continuing need for inhibitors of KSP which can be used in the treatment of a variety of diseases.

### SUMMARY OF THE INVENTION

The invention is directed to certain biphenyl compounds. Specifically, the invention is directed to compounds according to Formula I: 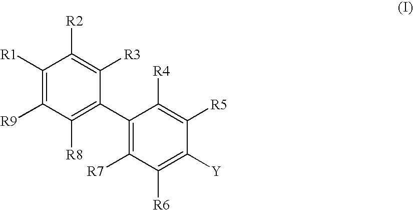
wherein R1, R2, R3, R4, R5, R6, R7, R8, R9, and Y are as defined below, and to pharmaceutically-acceptable salts thereof.

The compounds of the invention are KSP inhibitors, particularly human KSP inhibitors, and can be useful for the treatment for a variety of diseases and conditions, such as cancer, hyperplasias, restenosis, cardiac hypertrophy, immune disorders, fungal disorders, and inflammation. Accordingly, the invention is further directed to pharmaceutical compositions comprising a compound of the invention. The invention is still further directed to methods of inhibiting KSP and treatment of conditions associated therewith using a compound of the invention or a pharmaceutical composition comprising a compound of the invention.

In an additional aspect, the invention provides methods of screening for compounds that will bind to a KSP kinesin, for example compounds that will displace or compete with the binding of the compounds of the invention. The methods comprise combining a labeled compound of the invention, a KSP kinesin, and at least one candidate agent and determining the binding of the candidate bioactive agent to the KSP kinesin.

In a further aspect, the invention provides methods of screening for modulators of KSP kinesin activity. The methods comprise combining a compound of the invention, a KSP kinesin, and at least one candidate agent and determining the effect of the candidate bioactive agent on the KSP kinesin activity.

### DETAILED DESCRIPTION OF THE INVENTION

In describing the invention, chemical elements are identified in accordance with the Periodic Table of the Elements. Abbreviations and symbols utilized herein are in accordance with the common usage of such abbreviations and symbols by those skilled in the chemical and biological arts.

Specifically, the following abbreviations have the indicated meanings throughout: Ac represents acetyl, BNB represents 4-bromomethyl-3-nitrobenzoic acid, Boc represents t-butyloxy carbonyl, br represents broad, Bu represents butyl, c-represents cyclo, CBZ represents carbobenzoxy represents benzyloxycarbonyl, d represents doublet, DBU represents diazabicyclo[5.4.0]undec-7-ene, DCM represents dichloromethane represents methylene chloride represents CH₂Cl₂, DCE represents dichloroethylene, DEAD represents diethyl azodicarboxylate, DIC represents diisopropylcarbodiimide, DIEA represents N,N-diisopropylethyl amine, DMAP represents 4-N,N-dimethylaminopyridine, DMF represents N,N-dimethylformamide, DMSO represents dimethyl sulfoxide, DVB represents 1,4-divinylbenzene, EEDQ represents 2-ethoxy-1-ethoxycarbonyl-1,2-dihydroquinoline, ESMS represents electrospray mass spectrometry, Et represents ethyl, Fmoc represents 9-fluorenylmethoxycarbonyl, GC represents gas chromatography, HATU represents O-(7-Azabenzotriazol-1-yl)-1,1,3,3-tetramethyluronium, hexafluorophosphate, H MDS represents hexamethyldisilazane, HOAc represents acetic acid, HOBt represents hydroxybenzotriazole, HPLC represents high pressure liquid chromatography, m represents multiplet, Me represents methyl, Ms represents methanesulfonyl, MHz represents megahertz, MTBE represents methyl t-butyl ether, NMO represents N-methylmorpholine oxide, NMR represents nuclear magnetic resonance, PEG represents polyethylene glycol, Ph represents phenyl, PhOH represents phenol, PfP represents pentafluorophenol, PPTS represents pyridinium p-toluenesulfonate, Py represents pyridine, PyBroP represents bromo-tris-pyrrolidino-phosphonium hexafluorophosphate, rt or RT represent room temperature, sat'd represents saturated, s represents singlet, s-represents secondary, t represents triplet, t-represents tertiary, TBDMS represents t-butyldimethylsilyl, TES represents triethylsilyl, TFA represents trifluoroacetic acid, THF represents tetrahydrofuran, TMOF represents trimethyl orthoformate, TMS represents trimethylsilyl, tosyl represents p-toluenesulfonyl, and Trt represents triphenylmethyl.

Terms and Definitions

“Alkyl” refers to a saturated or unsaturated hydrocarbon chain having from 1 to 12 member atoms. Alkyl groups may be optionally substituted with one or more substituents as defined herein. Use of the prefix “C₁-Cₓ” with alkyl refers to an alkyl group having from 1 to x member atoms, where x is an integer from 2 to 12. For example, C₁-C₆ alkyl refers to an alkyl group having from 1 to 6 member atoms. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl. As stated above, alkyl includes unsaturated hydrocarbon chains. Thus, alkenyl and alkynyl are subsets of alkyl. “Alkenyl” refers to an unsaturated hydrocarbon chain having from 2 to 12 member atoms and having one or more carbon-carbon double bonds within the chain. In certain embodiments alkenyl groups have one carbon-carbon double bond within the chain. In other embodiments, alkenyl groups have more than one carbon-carbon double bond within the chain. Alkenyl includes ethylenyl, propenyl, butenyl, pentenyl, and hexenyl. “Alkynyl” refers to an unsaturated hydrocarbon chain having from 2 to 12 member atoms and having one or more carbon-carbon triple bonds within the chain. In certain embodiments alkynyl groups have one carbon-carbon triple bond within the chain. In other embodiments, alkynyl groups have more than one carbon-carbon triple bond within the chain. For the sake of clarity, unsaturated hydrocarbon chains having one or more carbon-carbon triple bond within the chain and one or more carbon-carbon double bond within the chain are alkynyl groups. Alkynyl includes ethynyl, propynyl, butynyl, pentynyl, and hexynyl.

“Antimitotic” refers to a compound that inhibits or prevents mitosis, for example, by causing metaphase arrest. Some antitumour compounds block proliferation and are considered antimitotics.

“Aryl” means phenyl or napthyl. Aryl groups may be optionally substituted with one or more substituents as defined herein.

“Cycloalkyl” refers to a saturated or unsaturated hydrocarbon ring having from 3 to 6 member atoms. Cycloalkyl groups are not aromatic. Cycloalkyl groups are monocyclic ring systems. Cycloalkyl groups may be optionally substituted with one or more substituents as defined herein. Use of the prefix “C₃-Cₓ” with cycloalkyl refers to a cycloalkyl group having from 3 to x member atoms, where x is an integer from 4 to 6. For example, C₃-C₆ cycloalkyl refers to a cycloalkyl group having from 3 to 6 member atoms. Cycloalkyl includes cyclopropyl, cyclobutyl, cyclopentyl, and cyclohexyl. As stated above, cycloalkyl includes unsaturated hydrocarbon rings. Thus, cycloalkenyl is a subset of cycloalkyl. “Cycloalkenyl” refers to an unsaturated hydrocarbon ring having from 3 to 6 member atoms and having a carbon-carbon double bond within the ring. In certain embodiments cycloalkenyl groups have one carbon-carbon double bond within the ring. In other embodiments, cycloalkenyl groups have more than one carbon-carbon double bond within the ring. However, cycloalkenyl rings are not aromatic. Cycloalkenyl groups are monocyclic ring systems. Cycloalkenyl includes cyclopropenyl, cyclobutenyl, cyclopentenyl, and cyclohexenyl.

“Enantiomerically enriched” refers to products whose enantiomeric excess is greater than zero. For example, enantiomerically enriched refers to products whose enantiomeric excess is greater than 50% ee, greater than 75% ee, and greater than 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that is constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 99% ee.

“Half-life” (or “half-lives”) refers to the time required for half of a quantity of a substance to be converted to another chemically distinct species in vitro or in vivo.

“Halo” refers to the halogen radical fluoro, chloro, bromo, or iodo.

“Haloalkyl” refers to an alkyl group wherein at least one hydrogen atom attached to a member atom within the alkyl group is replaced with halo.

“Heteroatom” refers to a nitrogen, sulphur, or oxygen atom.

“Heterocycloalkyl” refers to a saturated or unsaturated monocyclic ring having from 5 to 7 member atoms and containing from 1 to 3 heteroatoms as member atoms in the ring. Heterocycloalkyl rings are not aromatic. Heterocycloalkyl groups containing more than one heteroatom may contain different heteroatoms. Heterocycloalkyl groups may be optionally substituted with one or more substituents as defined herein. In certain embodiments, heterocycloalkyl is saturated. In other embodiments, heterocycloalkyl is unsaturated but not aromatic. Heterocycloalkyl includes pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be unsubstituted or substituted with one or more substituents as defined herein. “Substituted” in reference to a group indicates that a hydrogen atom attached to a member atom within a group is replaced. It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, cyclization, or elimination). In certain embodiments, a single atom may be substituted with more than one substituent as long as such substitution is in accordance with the permitted valence of the atom. Suitable substituents are defined herein for each substituted or optionally substituted group.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Compounds

The invention is directed to compounds having the following structure: 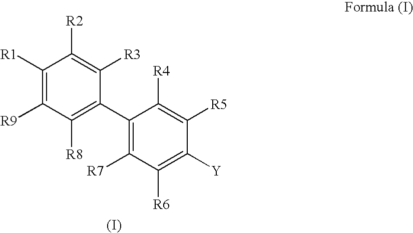
wherein:

R1 is selected from the group consisting of: NR10C(X)Z, H, halo, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl, wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and

wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO2NRbRe, N(Rb)SO2Re, C₁-C₃ alkyl and C₁-C₃ haloalkyl;

X is O or S;

Z is H or NHR11;

R2 is selected from the group consisting of: NR10C(X)Z, H, halo, CN, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and

wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;

provided that one and only one of R1 and R2 is NR10C(X)Z;

R3, R4, R7, and R8, are each independently selected from the group consisting of: H, halo, OH, CN, NO₂, NR12R13, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and

wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;

R5, R6, and R9 are each independently selected from the group consisting of: H, halo, CN, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and

wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;

R10 is selected from the group consisting of: H, OH, cyclopropyl, and C₁-C₃ alkyl;

R11 is selected from the group consisting of: H, ORf, optionally substituted C₁-C₅ alkyl, optionally substituted C₁-C₅ haloalkyl, optionally substituted C₃-C₆ cycloalkyl,

wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl, and

wherein said C₃-C₆ cycloalkyl is optionally substituted with one or more substituent selected from the group consisting of: ORa, NRbRc, C(O)ORa, C(O)NRbRc, N(Rb)C(O)Re, SO₂NRbRe, N(Rb)SO₂Re, C₁-C₃ alkyl and C₁-C₃ haloalkyl;

R12 is selected from the group consisting of H, C₁-C₃ alkyl, and cyclopropyl;

R13 is selected from the group consisting of: H, ORf, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, C₃-C₆ cycloalkyl, and heterocycloalkyl, and

wherein said C₃-C₆ cycloalkyl and heterocycloalkyl are optionally substituted with one or more substituent selected from the group consisting of: ORa, NRbRc, C(O)ORa, C(O)NRbRc, N(Rb)C(O)Re, SO₂NRbRe, —N(Rb)SO₂Re, C₁-C₃ alkyl and C₁-C₃ haloalkyl;

R14 is selected from the group consisting of: H, optionally substituted C₁-C₅ alkyl, optionally substituted C₁-C₅ haloalkyl, and optionally substituted C₃-C₆ cycloalkyl,

wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl, and

wherein said C₃-C₆ cycloalkyl is optionally substituted with one or more substituent selected from the group consisting of: ORa, NRbRc, C(O)ORa, C(O)NRbRc, N(Rb)C(O)Re, SO₂NRbRe, —N(Rb)SO₂Re, C₁-C₃ alkyl and C₁-C₃ haloalkyl;

when Z is NHR11Y is selected from the group consisting of: halo, OCF₃, S(O)ₙCF₃, optionally substituted C₁-C₅ alkyl, optionally substituted C₁-C₅ haloalkyl, and C(R15)(R16)(CF₃),

wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl,

when Z is H Y is selected from the group consisting of: OCF₃, S(O)ₙCF₃, optionally substituted C₁-C₄ alkyl, optionally substituted C₁-C₅ haloalkyl, and C(R15)(R16)(CF₃),

wherein said C₁-C₄ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl,

or when Z is H or NHR11Y and either R5 or R6 taken together with the carbon atoms to which they are attached form a ring having from 5 to 7 member atoms wherein said ring optionally contains 1 or 2 heteroatoms as member atoms, said ring is saturated or unsaturated, and said ring is optionally substituted with one or more substituent selected from the group consisting of: halo, ORa, CN, NR12R13, optionally substituted C₁-C₅ alkyl, and optionally substituted C₁-C₅ haloalkyl,

wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl;

R15 and R16 taken together with the carbon to which they are attached form a ring having from 3 to 6 member atoms wherein said ring optionally contains from 1 to 3 heteroatoms as member atoms, said ring is saturated or unsaturated, and said ring is optionally substituted with one or more substituent selected from the group consisting of: halo, —ORa, —CN, optionally substituted C₁-C₅ alkyl, and optionally substituted C₁-C₅ haloalkyl;

n is 0, 1, or 2;

Ra is selected from the group consisting of: H, optionally substituted C₁-C₅ alkyl, optionally substituted C₁-C₅ haloalkyl, and optionally substituted C₃-C₆ cycloalkyl,

wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and

wherein said C₃-C₆ cycloalkyl is optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;

Rb is selected from the group consisting of: H, C₁-C₃ alkyl, and cyclopropyl;

Rc is selected from the group consisting of: H, —ORd, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and

wherein said C₃-C₆ cycloalkyl and heterocycloalkyl are optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;

Rd is selected from the group consisting of: H and C₁-C₃ alkyl;

Re is selected from the group consisting of: H, optionally substituted C₁-C₅ alkyl, optionally substituted C₁-C₅ haloalkyl, optionally substituted C₃-C₆ cycloalkyl, and optionally substituted heterocycloalkyl,

wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and

wherein said C₃-C₆ cycloalkyl and heterocycloalkyl are optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;

Rf is selected from the group consisting of: H, optionally substituted C₁-C₅ alkyl, optionally substituted C₁-C₅ haloalkyl, and optionally substituted C₃-C₆ cycloalkyl,

wherein said C₁-C₅ alkyl and C₁-C₅ haloalkyl are optionally substituted with one or more substituent selected from the group consisting of: ORd, NRdRd, C(O)OH, C(O)NRdRd, N(Rd)C(O)Rd, SO₂NRdRd, N(Rd)SO₂Rd, and C₃-C₆ cycloalkyl, and

wherein said C₃-C₆ cycloalkyl is optionally substituted with one or more substituent selected from the group consisting of: ORd, NRdRd, C(O)OH, C(O)NRdRd, N(Rd)C(O)Rd, SO₂NRdRd, N(Rd)SO₂Rd, C₁-C₃ alkyl and C₁-C₃ haloalkyl.

The meaning of any functional group or substituent thereon at any one occurrence in Formula I, or any subformula thereof, is independent of its meaning, or any other functional group's or substituent's meaning, at any other occurrence, unless stated otherwise.

The compounds according to Formula I may contain one or more asymmetric centers (also referred to as a chiral center) and may, therefore, exist as individual enantiomers, diasteriomers, or other stereoisomeric forms, or as mixtures thereof. Chiral centers, such as chiral carbon atoms, may also be present in a substituent such as an alkyl group. Where the stereochemistry of a chiral center present in Formula I, or in any chemical structure illustrated herein, is not specified the structure is intended to encompass any stereoisomer and all mixtures thereof. Thus, compounds according to Formula I containing one or more chiral centers may be used as racemic mixtures, enantiomerically enriched mixtures, or as enantiomerically pure individual stereoisomers.

Individual stereoisomers of a compound according to Formula I which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. Likewise, all tautomeric forms are also included in Formula I whether such tautomers exist in equilibrium or predominately in one form.

The skilled artisan will appreciate that pharmaceutically-acceptable salts of the compounds according to Formula I may be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula I.

As used herein, the term “pharmaceutically-acceptable salts” refers to salts that retain the desired biological activity of the subject compound and exhibit minimal undesired toxicological effects. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group. Suitable pharmaceutically-acceptable salts include salts of such acidic functional groups. Representative salts include pharmaceutically-acceptable metal salts such as sodium, potassium, lithium, calcium, magnesium, aluminum, and zinc salts; carbonates and bicarbonates of a pharmaceutically-acceptable metal cation such as sodium, potassium, lithium, calcium, magnesium, aluminum, and zinc; pharmaceutically-acceptable organic primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula I may contain a basic functional group and are therefore capable of forming pharmaceutically-acceptable acid addition salts by treatment with a suitable acid. Suitable acids include pharmaceutically-acceptable inorganic acids and pharmaceutically-acceptable organic acids. Representative pharmaceutically-acceptable acid addition salts include hydrochloride, hydrobromide, nitrate, methylnitrate, sulfate, bisulfate, sulfamate, phosphates acetate, hydroxyacetate, phenylacetate, propionate, butyrate, isobutyrate, valerate, maleate, hydroxymaleate, acrylate, fumarate, malate, tartrate, citrate, salicylate, praminosalicyclate, glycollate, lactate, heptanoate, phthalate, oxalate, succinate, benzoate, oacetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), paminobenzenesulfonate, p-toluenesulfonate (tosylate), and napthalene-2-sulfonate.

As used herein, the term “compounds of the invention” means both the compounds according to Formula I and the pharmaceutically-acceptable salts thereof. The term “a compound of the invention” also appears herein and refers to both a compound according to Formula I and its pharmaceutically-acceptable salts.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

Nomenclature

The compounds of Formula I can be named and numbered (e.g., using ACD/Name add-in for ISIS/Draw version 6.02) as described below.

For example, the compound 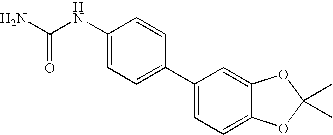
i.e., the compound of Formula I where R1 is —NHC(O)NH₂, R2-R4 and R6-R9 are hydrogen, and R5 and Y taken together with the carbon atoms to which they are attached is 2,2-dimethyl-1,3-dioxole, can be named N-[4-(2,2-dimethyl-1,3-benzodioxol-5-yl)phenyl]urea; 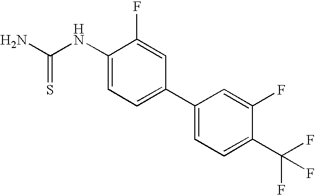
i.e., the compound of Formula I where R1 is —NHC(S)NH₂, R2 is fluoro, R3-R4 are hydrogen, R5 is fluoro, R6-R9 are hydrogen, and Y is trifluoromethyl, can be named N-[3,3′-difluoro-4′-(trifluoromethyl)-4-biphenylyl]thiourea; 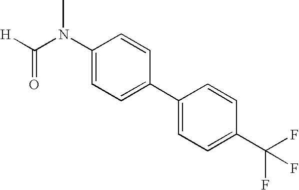
i.e., the compound of Formula I where R1 is —N(Me)C(O)H, R2-R9 are hydrogen, and Y is trifluoromethyl, can be named methyl[4′-(trifluoromethyl)-4-biphenylyl]formamide.

In certain embodiments of the compounds of Formula I, R1 is NR10C(X)Z or H. In certain further embodiments, R1 is NR10C(X)Z.

In certain embodiments of the compounds of Formula I, X is O. In certain other embodiments, X is S.

In certain embodiments of the compounds of Formula I, R10 is H, OH, or C₁-C₃ alkyl. In certain further embodiments, R10 is H, OH or methyl. In still further embodiments, R10 is H.

In certain embodiments of the compounds of Formula I, Z is H. In certain other embodiments, Z is NHR11.

In certain embodiments of the compounds of Formula I, R11 is H, ORf, or optionally substituted C₁-C₅ alkyl. In certain further embodiments, R11 is H or OH. In still further embodiments, R11 is H.

In certain embodiments of the compounds of Formula I, R2 is H, halo, NR12R13, OR14, or optionally substituted C₁-C₅ alkyl. In certain further embodiments, R2 is H, halo, NH₂, or CH₃. In still further embodiments, R2 is H, halo, or NH₂. In still further embodiments, R2 is H, fluoro, or NH₂. In still further embodiments, R2 is H or fluoro.

In certain embodiments of the compounds of Formula I, R3, R4, R7, and R8 are each independently H, halo, NR12R13, optionally substituted C₁-C₅ alkyl, or optionally substituted C₁-C₅ haloalkyl. In certain further embodiments, R3, R4, R7, and R8 are each independently H, halo, NH₂, or CH₃. In still further embodiments, R3, R4, R7, and R8 are each independently H or halo. In still further embodiments, R3, R4, R7, and R8 are each independently H or fluoro. In still further embodiments, R3, R4, R7, and R8 are each H.

In certain embodiments of the compounds of Formula I, R5 and R6 are each independently H, halo, NR12R13, optionally substituted C₁-C₅ alkyl, or optionally substituted C₁-C₅ haloalkyl. In certain further embodiments, R5 and R6 are each independently H, halo, NR12R13, CF₃ or CH₃. In still further embodiments, R5 and R6 are each independently H, halo, or CF₃. In still further embodiments, R5 and R6 are each independently H or fluoro.

In certain embodiments of the compounds of Formula I, R9 is H, halo, NR12R13, OR14, or optionally substituted C₁-C₅ alkyl. In certain further embodiments, R9 is H, halo, NH₂, or CH₃. In still further embodiments, R9 is H or halo. In still further embodiments, R9 is H or fluoro. In still further embodiments, R9 is H.

In certain embodiments of the compounds of Formula I, when Z is NHR11Y is halo. In certain further embodiments, Y is bromo, chloro, or fluoro. In still further embodiments, Y is bromo or chloro. In still further embodiments, Y is bromo. In certain other embodiments, Y is OCF₃. In certain other embodiments, Y is S(O)ₙCF₃. In certain further embodiments, n is 2. In certain other embodiments, Y is optionally substituted C₁-C₅ alkyl. In certain further embodiments, Y is isobutyl, t-butyl, or isopropyl. In still further embodiments, Y is t-butyl, or isopropyl. In certain other embodiments, Y is optionally substituted C₁-C₅ haloalkyl. In certain further embodiments, Y is CF₃. In certain other embodiments, Y is C(R15)(R16)(CF₃).

In certain embodiments of the compounds of Formula I, when Z is H Y is OCF₃. In certain other embodiments, Y is S(O)ₙCF₃. In certain further embodiments, n is 2. In certain other embodiments, Y is optionally substituted C₁-C₄ alkyl. In certain further embodiments, Y is isobutyl, t-butyl, or isopropyl. In still further embodiments, Y is t-butyl, or isopropyl. In certain other embodiments, Y is optionally substituted C₁-C₅ haloalkyl. In certain further embodiments, Y is CF₃. In certain other embodiments, Y is C(R15)(R16)(CF₃).

In certain embodiments of the compounds of Formula I, when Z is H or NHR11Y and either R5 or R6 taken together with the carbon atoms to which they are attached form a ring as defined above. In certain further embodiments, the ring has 5 or 6 member atoms. In certain further embodiments, the ring is saturated or unsaturated. In still further embodiments, the ring has 1 or 2 heteroatoms as member atoms selected from O or S. In still further embodiments, the ring has two heteroatoms as member atoms. In still further embodiments, the ring has two O atoms as member atoms. In certain further embodiments the ring is substituted with from 1 to 4 substituents selected from the group consisting of: halo, C₁-C₃ alkyl, and C₁-C₃ haloalkyl; in particular embodiments the substituents are selected from halo and C₁-C₃ alkyl. In still further embodiments, the ring is substituted with 1 or 2 substituents selected from fluoro, methyl and/or trifluoromethyl. In other further embodiments when the ring has 6 member atoms, the ring is substituted with 4 fluoro groups. In particular embodiments, the ring comprises 1,3-dioxole, dihydrofuran, 4-H-1,3-dioxin or 2,3-dihydro-1,4-dioxin, e.g., 2,2-difluoro-1,3-dioxole or 2,2,3,3-tetrafluoro-2,3-dihydro-1,4-dioxin.

Particular examples of compounds of the present invention include:
N-[4′-(trifluoromethyl)-3-biphenylyl]urea;
N-(4′-methyl-4-biphenylyl)urea;
N-[4′-(isopropyl)-4-biphenylyl]urea;
N-[4′-(t-butyl)-4-biphenylyl]urea;
N-{4′-[(trifluoromethyl)thio]-4-biphenyl}urea;
N-[4′-(trifluoromethyl)-4-biphenylyl]urea;
N-[3′-fluoro-4′-(trifluoromethyl)-4-biphenylyl]urea;
N-{4′-[(trifluoromethyl)sulfonyl]-4-biphenylyl}urea;
N-methyl-N-[4′-(trifluoromethyl)-4-biphenylyl]urea;
N-[3-bromo-5-fluoro-4′-(trifluoromethyl)-4-biphenylyl]urea;
N-[4-(2,2,3,3-tetrafluoro-2,3-dihydro-1,4-benzodioxin-6-yl)phenyl]urea;
N-[3-fluoro-4′-(trifluoromethyl)-4-biphenylyl]urea;
N-[3-cyano-4′-(trifluoromethyl)-4-biphenylyl]urea;
N-[3-methoxy-4′-(trifluoromethyl)-4-biphenylyl]urea;
N-[4-(2,2-difluoro-1,3-benzodioxol-5-yl)phenyl]urea;
N-[4-(2,2-difluoro-1,3-benzodioxol-5-yl)-2-fluorophenyl]urea;
N-[2′-chloro-4′-(trifluoromethyl)-4-biphenylyl]urea;
N-[2′-chloro-3-fluoro-4′-(trifluoromethyl)-4-biphenylyl]urea;
N-{4′-[(trifluoromethyl)sulfinyl]-4-biphenylyl}urea;
N-[4-(2,2,4,4-tetrafluoro-4H-1,3-benzodioxin-6-yl)phenyl]urea;
N-[4-(2,2-dimethyl-1,3-benzodioxol-5-yl)phenyl]urea;
N-[3,3′-difluoro-4′-(trifluoromethyl)-4-biphenylyl]urea;
N-[3-hydroxy-4′-(trifluoromethyl)-4-biphenylyl]urea;
N-[3-amino-4′-(trifluoromethyl)-4-biphenylyl]urea;
N-{4′-[3-(trifluoromethyl)-3-diaziridinyl]-4-biphenylyl}urea;
N-{4′-[3-(trifluoromethyl)-3H-diazirin-3-yl]-4-biphenylyl}urea;
N-hydroxy-N′-[4′-(trifluoromethyl)-4-biphenylyl]urea;
N-(3′,4′-dichloro-3-fluoro-4-biphenylyl)urea;
N-(3-fluoro-4′-propyl-4-biphenylyl)urea;
N-[4-(2,3-dihydro-1-benzofuran-5-yl)-2-fluorophenyl]urea;
N-(3-fluoro-3′,4′-dimethyl-4-biphenylyl)urea;
N-(3-fluoro-4′-isobutyl-4-biphenylyl)urea;
N-(3,3′,4′,5′-tetrafluoro-4-biphenylyl)urea;
N-(4′-bromo-3,3′-difluoro-4-biphenylyl)urea;
N-(4′-ethenyl-3-fluoro-4-biphenylyl)urea;
N-[4′-(trifluoromethyl)-4-biphenylyl]thiourea;
N-[3,3′-difluoro-4′-(trifluoromethyl)-4-biphenylyl]thiourea;
[4′-(trifluoromethyl)-4-biphenylyl]formamide;
[3′-fluoro-4′-(trifluoromethyl)-4-biphenylyl]formamide;
[3,3′-difluoro-4′-(trifluoromethyl)-4-biphenylyl]formamide;
[3-fluoro-4′-(trifluoromethyl)-4-biphenylyl]formamide;
{4′-[(trifluoromethyl)sulfonyl]-4-biphenylyl}formamide;
hydroxy[4′-(trifluoromethyl)-4-biphenylyl]formamide;
[4′-(trifluoromethyl)-4-biphenylyl]thioformamide;
[3-amino-5-fluoro-4′-(trifluoromethyl)-4-biphenylyl]formamide; and
methyl[4′-(trifluoromethyl)-4-biphenylyl]formamide.
   Compound Preparation

The compounds of the invention may be prepared according to the general process outlined below in Schemes 1-5 and described in the Examples. The starting materials shown in the schemes are commercially available, such as from Aldrich Chemical Company, Milwaukee, Wis., or may be readily prepared from commercially available starting materials using methods known to those skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

Compounds of formula (I) can be readily prepared via a cross-coupling reaction between a suitable aryl halide and a suitable aryl organometallic agent under standard conditions (Scheme 1). The coupling of suitable aryl halides, such as when X is iodine, bromine, or chlorine, and suitable aryl organometallic agents, such as when M is (functionalized) boron, magnesium, or tin, is discussed in depth in the literature. Aryl halides and, as an example, aryl boronic acids/esters are either commercially available, reported in the literature, or can be prepared following literature procedures by those skilled in the art.

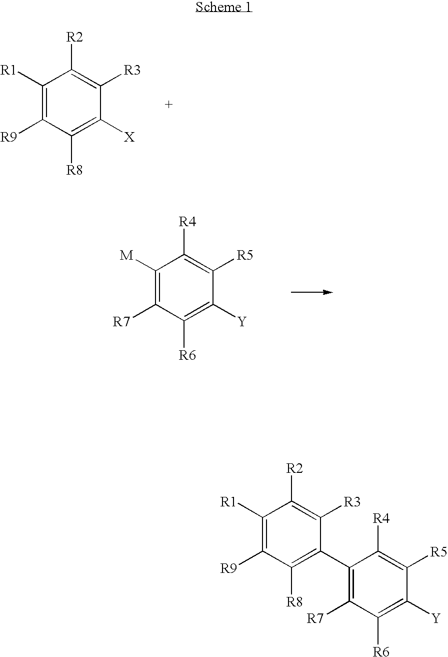

Suzuki cross-coupling of an aryl halide (such as a functionalized bromoaniline) with a functionalized aryl boronic acid using a palladium catalyst (typically tetrakis(triphenylphospine)palladium(0)) in the presence of a base (such as potassium carbonate solution) and a suitable solvent (such as N,N-dimethylformamide) at elevated temperatures (for example, 100° C.) affords a functionalized biarylaniline (Scheme 2). This reaction can also be performed when the coupling partners are switched, i.e. the aniline moiety is the boronic acid and the aryl boronic acid is the aryl halide.

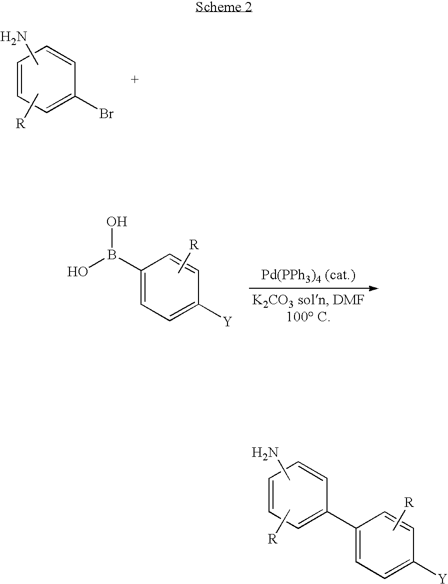

Such biarylanilines can then be transformed to ureas or formamides under standard conditions using functionalized isocyanates or formates. For example, reaction of a functionalized biarylaniline with an isocyanate in a suitable solvent (such as dichloromethane) and optionally a suitable base (such as pyridine) at room temperature or elevated temperature provides a functionalized biarylurea (Scheme 3). Similarly, reaction of a functionalized biarylaniline with a formate (such as p-nitrophenyl formate) in a suitable solvent (such as dichloromethane) and in the presence of a suitable base (such as pyridine) provides a biarylformamide.

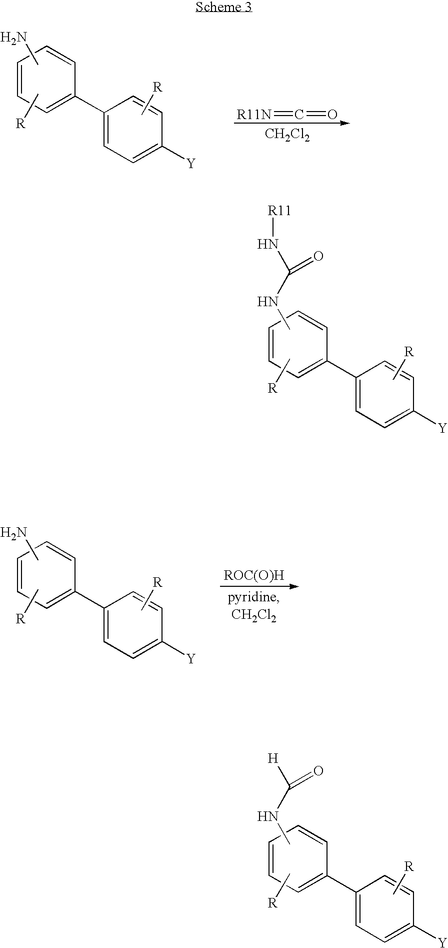

Such biarylanilines can also be transformed into thioureas under standard conditions using ammonium thiocyanate. For example, reaction of a functionalized biarylaniline with ammonium thiocyanate in a suitable solvent (such as tetrahydrofuran or water) with acid (such as 1N hydrochloric acid) under reflux provides a functionalized biarylthiourea (Scheme 4).

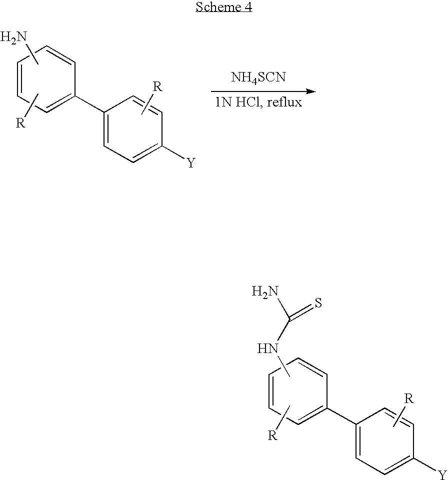

Alternatively, biaryl formation can occur at a later step using more highly functionalized coupling partners. For example, Suzuki cross-coupling reaction of a functionalized aryl halide (such as an aryl bromide) with a functionalized boronic acid/ester (such as an arylurea boronic ester) using a palladium catalyst (typically tetrakis(triphenylphosphine)palladium(0)) in the presence of a base (such as potassium carbonate solution) and a suitable solvent (such as N,N-dimethylformamide) at elevated temperatures (for example, 100° C.) affords a biaryl product such as a functionalized biphenylurea (Scheme 5). This reaction can also be performed when the coupling partners are switched, i.e. the functionalized boronic acid/ester is instead the aryl halide and the functionalized aryl halide is instead the boronic acid/ester.

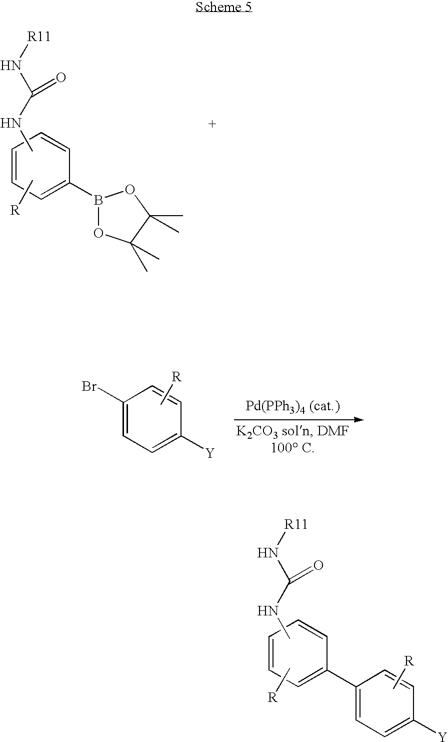

Thus, compounds of formula (I) may be prepared by a method comprising either:
a) reacting a compound of the formula (II) 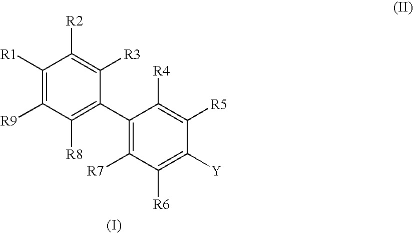
with either an isocyanate, a formate, or ammonium thiocyanate;
wherein one and only one of R1 and R2 are NH₂ and R3-R9 and Y are as defined above, and wherein the compound of formula (II) is optionally prepared by either

(i) reacting a compound of formula (a):

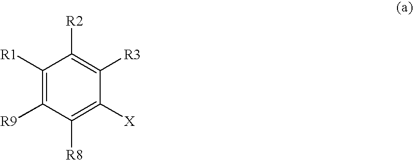

with a compound of formula (b):

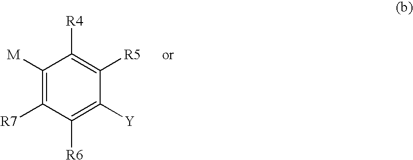

(ii) reacting a compound of formula (c):

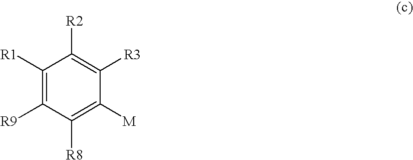

with a compound of formula (d): 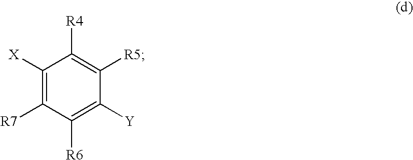
wherein one and only one of R1 and R2 are NH₂, R3-R9 and Y are as defined above, X is halo, and M is functionalized boron, magnesium, or tin; or
b) reacting a compound of formula (III) 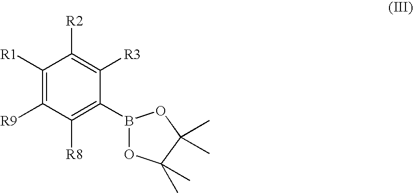
with a compound of formula (IV) 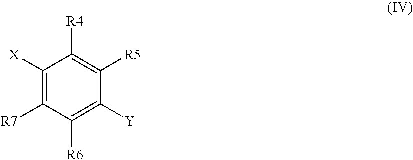
wherein R1-R9 and Y are as defined above, and X is halo.
Methods of Use

The present invention is directed to a class of novel compounds that are modulators, particularly inhibitors, of mitotic kinesins. By inhibiting or modulating mitotic kinesins, but not other kinesins (e.g., transport kinesins), specific inhibition of cellular proliferation is accomplished. Thus, the present invention makes use of the finding that perturbation of mitotic kinesin function causes malformation or dysfunction of mitotic spindles, frequently resulting in cell cycle arrest and cell death.

The methods of inhibiting a human KSP kinesin comprise contacting an inhibitor of the invention with a KSP kinesin, particularly human KSP kinesins, including fragments and variants of KSP. The inhibition can be of the ATP hydrolysis activity of the KSP kinesin and/or the mitotic spindle formation activity, such that the mitotic spindles are disrupted. Meiotic spindles may also be disrupted.

The compounds of the invention are inhibitors of mitotic kinesins, in particular KSP, and are therefore useful for the treatment of disorders associated with cell proliferation. Traditionally, dramatic improvements in the treatment of cancer, one type of cell proliferative disorder, have been associated with identification of therapeutic agents acting through novel mechanisms. Examples of this include not only the taxane class of agents that appear to act on microtubule formation, but also the camptothecin class of topoisomerase I inhibitors. The compounds of the invention can differ in their selectivity and can be used to treat diseases of proliferating cells, including, but not limited to cancer, hyperplasias, restenosis, cardiac hypertrophy, immune disorders, fungal disorders and inflammation.

Once made, the compounds of the invention find use in a variety of applications. As will be appreciated by those skilled in the art, mitosis may be altered in a variety of ways; that is, one can affect mitosis either by increasing or decreasing the activity of a component in the mitotic pathway. Stated differently, mitosis may be affected (e.g., disrupted) by disturbing equilibrium, either by inhibiting or activating certain components. Similar approaches may be used to alter meiosis.

In certain embodiments, the compounds of the invention are used to modulate mitotic spindle formation, thus causing prolonged cell cycle arrest in mitosis. By “modulate” herein is meant altering mitotic spindle formation, including increasing and decreasing spindle formation. By “mitotic spindle formation” herein is meant organization of microtubules into bipolar structures by mitotic kinesins. By “mitotic spindle dysfunction” herein is meant mitotic arrest and monopolar spindle formation.

The compounds of the invention are useful to bind to and/or modulate the activity of a mitotic kinesin, KSP. In certain embodiments, the KSP is human KSP, although KSP kinesins from other organisms may also be used. In this context, modulate means either increasing or decreasing spindle pole separation, causing malformation, i.e., splaying, of mitotic spindle poles, or otherwise causing morphological perturbation of the mitotic spindle. Also included within the definition of KSP for these purposes are variants and/or fragments of KSP. See U.S. Pat. Nos. 6,414,121 and 6,437,115, hereby incorporated by reference in their entirety. In addition, other mitotic kinesins may be used in the present invention. However, the compounds of the invention have been shown to have specificity for KSP.

The compounds of the invention can be used to treat cellular proliferation diseases. Disease states which can be treated by the compounds of the invention include, but are not limited to, cancer (further discussed below), autoimmune disease, fungal disorders, arthritis, graft rejection, inflammatory bowel disease, proliferation induced after medical procedures, including, but not limited to, surgery, angioplasty, and the like. It is appreciated that in some cases the cells may not be in a hyper or hypo proliferation state (abnormal state) and still require treatment. Thus, in certain embodiments, the invention includes application to cells or individuals afflicted or impending affliction with any one of these disorders or states.

The compounds of the invention provided herein are particularly deemed useful for the treatment of cancer including solid tumors such as skin, breast, brain, cervical carcinomas, testicular carcinomas, etc. More particularly, cancers that may be treated using the compounds of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Accordingly, the compounds of the invention are administered to cells. By “administered” herein is meant administration of a therapeutically effective dose of a compound of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered. A “patient” for the purposes of the present invention includes both humans and other animals, particularly mammals, and other organisms. Thus the methods are applicable to both human therapy and veterinary applications. In certain embodiments the patient is a mammal, especially a human.

The compounds of the invention may be administered in a physiologically acceptable carrier to a patient, as described herein. Depending upon the manner of introduction, the compounds may be formulated in a variety of ways as discussed below. The concentration of the compound in the formulation may vary from about 0.1-99.9 wt. %. The compounds of the invention can be administered alone or in combination with other treatments, i.e., radiation, or other therapeutic agents, such as the taxane class of agents that appear to act on microtubule formation or the camptothecin class of topoisomerase I inhibitors. When so-used, other therapeutic agents can be administered before, concurrently (whether in separate dosage forms or in a combined dosage form), or after administration of the compound of the invention.

Assays

For assay of activity, generally either KSP or a compound of the invention is non-diffusably bound to an insoluble support having isolated sample receiving areas (e.g., a microtiter plate, an array, etc.). The insoluble support may be made of any composition to which the compounds can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. The particular manner of binding of the compound is not crucial so long as it is compatible with the reagents and overall methods of the invention, maintains the activity of the compound and is nondiffusable. Such methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

The compounds of the invention may be used on their own to modulate the activity of a mitotic kinesin, particularly KSP. In this embodiment, the compounds of the invention are combined with KSP and the activity of KSP is assayed. Kinesin activity is known in the art and includes one or more kinesin activities. Kinesin activities include the ability to affect ATP hydrolysis; microtubule binding; gliding and polymerization/depolymerization (effects on microtubule dynamics); binding to other proteins of the spindle; binding to proteins involved in cell-cycle control; serving as a substrate to other enzymes; such as kinases or proteases; and specific kinesin cellular activities such as spindle pole separation.

Methods of performing motility assays are well known to those of skill in the art. (See e.g., Hall, et al. (1996), Biophys. J., 71: 3467-3476, Turner et al., 1996, Anal. Biochem. 242 (1):20-5; Gittes et al., 1996, Biophys. J. 70(1): 418-29; Shirakawa et al., 1995, J. Exp. Biol. 198: 1809-15; Winkelmann et al., 1995, Biophys. J. 68: 2444-53; Winkelmann et al., 1995, Biophys. J. 68: 72S.)

Methods known in the art for determining ATPase hydrolysis activity also can be used. Preferably, solution based assays are utilized. U.S. application Ser. No. 09/314,464, filed May 18, 1999, issued as U.S. Pat. No. 6,410,254, hereby incorporated by reference in its entirety, describes such assays. Alternatively, conventional methods are used. For example, Pᵢ release from kinesin can be quantified. In one particular embodiment, the ATPase hydrolysis activity assay utilizes 0.3 M PCA (perchloric acid) and malachite green reagent (8.27 mM sodium molybdate 11, 0.33 mM malachite green oxalate, and 0.8 mM Triton X-100). To perform the assay, 10 μL of reaction is quenched in 90 μL of cold 0.3 M PCA. Phosphate standards are used so data can be converted to mM inorganic phosphate released. When all reactions and standards have been quenched in PCA, 100 μL of malachite green reagent is added to the relevant wells in e.g., a microtiter plate. The mixture is developed for 10-15 minutes and the plate is read at an absorbance of 650 nm. If phosphate standards were used, absorbance readings can be converted to mM Pᵢ and plotted over time. Additionally, ATPase assays known in the art include the luciferase assay.

ATPase activity of kinesin motor domains also can be used to monitor the effects of modulating agents. In one embodiment ATPase assays of kinesin are performed in the absence of microtubules. In another embodiment, the ATPase assays are performed in the presence of microtubules.

Different types of modulating agents can be detected in the above assays. In a particular embodiment, the effect of a modulating agent is independent of the concentration of microtubules and ATP. In another embodiment, the effect of the agents on kinesin ATPase can be decreased by increasing the concentrations of ATP, microtubules or both (i.e., the effect can be increased by decreasing the concentrations of ATP, microtubules or both). In yet another embodiment, the effect of the modulating agent is increased by increasing concentrations of ATP, microtubules or both.

Agents that modulate the biochemical activity of KSP in vitro may then be screened in vivo. Methods for such agents in vivo include assays of cell cycle distribution, cell viability, or the presence, morphology, activity, distribution, or amount of mitotic spindles. Methods for monitoring cell cycle distribution of a cell population, for example, by flow cytometry, are well known to those skilled in the art, as are methods for determining cell viability. See for example, U.S. patent application “Methods of Screening for Modulators of Cell Proliferation and Methods of Diagnosing Cell Proliferation States,” filed Oct. 22, 1999, Ser. No. 09/428,156, issued as U.S. Pat. No. 6,617,115, hereby incorporated by reference in its entirety.

In addition to the assays described above, microscopic methods for monitoring spindle formation and malformation are well known to those of skill in the art (see, e.g., Whitehead and Rattner (1998), J. Cell Sci. 111:2551-61; Galgio et al, (1996) J. Cell biol., 135:399-414).

The compounds of the invention inhibit the KSP kinesin. One measure of inhibition is IC₅₀, defined as the concentration of the compound at which the activity of KSP is decreased by fifty percent relative to a control. Preferred compounds have IC₅₀'s of less than about 1 mM, with preferred embodiments having IC₅₀'s of less than about 100 μM, with more preferred embodiments having IC₅₀'s of less than about 10 μM, with particularly preferred embodiments having IC₅₀'s of less than about 1 μM, and especially preferred embodiments having IC₅₀'s of less than about 100 nM, and more preferably less than about 10 nM. Measurement of IC₅₀ is done using an ATPase assay.

Another measure of inhibition is Kᵢ. For compounds with IC₅₀'s less than 1 μM, the Kᵢ or K_{d} is defined as the dissociation rate constant for the interaction of the compounds described herein with KSP. Preferred compounds have Kᵢ's of less than about 100 μM, with preferred embodiments having Kᵢ's of less than about 10 μM, with particularly preferred embodiments having Kᵢ's of less than about 1 μM, and especially preferred embodiments having Kᵢ's of less than about 100 nM. The Kᵢ for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + Kd ) - \sqrt{{( {E}_{0} + {I}_{0 } + Kd )}^{2} - 4 {E}_{0} {I}_{0}}}{2 {E}_{0}} ]$$

where V is the observed rate, Vₘₐₓ is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Preferred compounds have GI₅₀'s of less than about 1 mM. The level of preferability of embodiments is a function of their GI₅₀: those having GI₅₀'s of less than about 20 μM are more preferred; those having GI₅₀'s of 10 μM more so; those having GI₅₀ of less than about 1 μM more so. Measurement of GI₅₀ is done using a cell proliferation assay.

Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from about 0.1 to 99.9 wt. %, depending on the nature of the formulation.

The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when comingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company, e.g., 18th Edition, A. R. Gennaro, Editor, 1990), Remington: The Science and Practice of Pharmacy, (Lippincott Williams & Wilkins, e.g., 20th Edition, A. R. Gennaro, Editor, 2000), The Handbook of Pharmaceutical Additives (Gower Publishing Limited, e.g., compiled by Michael and Irene Ash, 1995), and The Handbook of Pharmaceutical Excipients (the American Pharmaceutical Association and the Pharmaceutical Press, e.g., 3rd Edition, A. H. Kibbe, Editor, 2000).

The pharmaceutical compositions of the invention are prepared using techniques and methods known to those skilled in the art. Some of the methods commonly used in the art are described in Remington's Pharmaceutical Sciences, supra.

Oral solid dosage forms such as tablets will typically comprise one or more pharmaceutically acceptable excipients, which may for example help impart satisfactory processing and compression characteristics, or provide additional desirable physical characteristics to the tablet. Such pharmaceutically acceptable excipients may be selected from diluents, binders, glidants, lubricants, disintegrants, colorants, flavorants, sweetening agents, polymers, waxes or other solubility-modulating materials.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy, supra., and include: alcohol, e.g., 5% alcohol (e.g., in dextrose and water (“D/W”) or D/W in normal saline solution (“NSS”), including in 5% dextrose and water (“D5/W”), or D5/W in NSS); synthetic amino acid such as Aminosyn, FreAmine, Travasol, e.g., 3.5 or 7; 8.5; 3.5, 5.5 or 8.5% respectively; ammonium chloride e.g., 2.14%; dextran 40, in NSS e.g., 10% or in D5/W e.g., 10%; dextran 70, in NSS e.g., 6% or in D5/W e.g., 6%; dextrose (glucose, D5/W) e.g., 2.5-50%; dextrose and sodium chloride e.g., 5-20% dextrose and 0.22-0.9% NaCl; lactated Ringer's (Hartmann's) e.g., NaCl 0.6%, KCl 0.03%, CaCl₂ 0.02%; lactate 0.3%; mannitol e.g., 5%, optionally in combination with dextrose e.g., 10% or NaCl e.g., 15 or 20%; multiple electrolyte solutions with varying combinations of electrolytes, dextrose, fructose, invert sugar Ringer's e.g., NaCl 0.86%, KCl 0.03%, CaCl₂ 0.033%; sodium bicarbonate e.g., 5%; sodium chloride e.g., 0.45, 0.9, 3, or 5%; sodium lactate e.g., 1/6 M; and sterile water for injection. The pH of such IV fluids may vary, and will typically be from 3.5 to 8 as known in the art.

Screening Methods

To employ the compounds of the invention in a method of screening for compounds that bind to KSP kinesin, the KSP is bound to a support, and a compound of the invention (which is an anti-mitotic agent) is added to the assay. Alternatively, the compound of the invention is bound to the support and KSP is added. Classes of compounds among which novel binding agents may be sought include specific antibodies, non-natural binding agents identified in screens of chemical libraries, peptide analogs, etc. Of particular interest are screening assays for candidate agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.) and the like.

The determination of the binding of the anti-mitotic agent to KSP may be done in a number of ways. In a particular embodiment, the anti-mitotic agent (the compound of the invention) is labeled, for example, with a fluorescent or radioactive moiety and binding determined directly. For example, this may be done by attaching all or a portion of KSP to a solid support, adding a labeled anti-mitotic agent (for example a compound of the invention in which at least one atom has been replaced by a detectable isotope), washing off excess reagent, and determining whether the amount of the label is that present on the solid support. Various blocking and washing steps may be utilized as is known in the art.

By “labeled” herein is meant that the compound is either directly or indirectly labeled with a label which provides a detectable signal, e.g., radioisotope, fluorescent tag, enzyme, antibodies, particles such as magnetic particles, chemiluminescent tag, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

In some embodiments, only one of the components is labeled. For example, the kinesin proteins may be labeled at tyrosine positions using ¹²⁵I, or with fluorophores. Alternatively, more than one component may be labeled with different labels; using ¹²⁵I for the proteins, for example, and a fluorophor for the anti-mitotic agents.

The compounds of the invention may also be used as competitors to screen for additional drug candidates. “Candidate bioactive agent” or “drug candidate” or grammatical equivalents as used herein describe any molecule, e.g., protein, oligopeptide, small organic molecule, polysaccharide, polynucleotide, etc., to be tested for bioactivity. They may be capable of directly or indirectly altering the cellular proliferation phenotype or the expression of a cellular proliferation sequence, including both nucleic acid sequences and protein sequences. In other cases, alteration of cellular proliferation protein binding and/or activity is screened. Screens of this sort may be performed either in the presence or absence of microtubules. In the case where protein binding or activity is screened, preferred embodiments exclude molecules already known to bind to that particular protein, for example, polymer structures such as microtubules, and energy sources such as ATP. Particular embodiments of assays herein include candidate agents which do not bind the cellular proliferation protein in its endogenous native state termed herein as “exogenous” agents. In another particular embodiment, exogenous agents further exclude antibodies to KSP.

Candidate agents can encompass numerous chemical classes, though typically they are organic molecules, preferably small organic compounds having a molecular weight of more than 100 and less than about 2,500 daltons. Candidate agents comprise functional groups necessary for structural interaction with proteins, particularly hydrogen bonding and lipophilic binding, and typically include at least an amine, carbonyl, hydroxyl, ether, or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

Competitive screening assays may be done by combining KSP and a drug candidate in a first sample. A second sample comprises a anti-mitotic agent, KSP and a drug candidate. This may be performed in either the presence or absence of microtubules. The binding of the drug candidate is determined for both samples, and a change, or difference in binding between the two samples indicates the presence of an agent capable of binding to KSP and potentially modulating its activity. That is, if the binding of the drug candidate is different in the second sample relative to the first sample, the drug candidate is capable of binding to KSP.

In a particular embodiment, the binding of the candidate agent is determined through the use of competitive binding assays. In this embodiment, the competitor is a binding moiety known to bind to KSP, such as an antibody, peptide, binding partner, ligand, etc. Under certain circumstances, there may be competitive binding as between the candidate agent and the binding moiety, with the binding moiety displacing the candidate agent.

In certain embodiments, the candidate agent is labeled. Either the candidate agent, or the competitor, or both, is added first to KSP for a time sufficient to allow binding, if present. Incubations may be performed at any temperature which facilitates optimal activity, typically between 4 and 40° C.

Incubation periods are selected for optimum activity, but may also be optimized to facilitate rapid high throughput screening. Typically between 0.1 and 1 hour will be sufficient. Excess reagent is generally removed or washed away. The second component is then added, and the presence or absence of the labeled component is followed, to indicate binding.

In particular embodiments, the competitor is added first, followed by the candidate agent. Displacement of the competitor is an indication the candidate agent is binding to KSP and thus is capable of binding to, and potentially modulating, the activity of KSP. In this embodiment, either component can be labeled. Thus, for example, if the competitor is labeled, the presence of label in the wash solution indicates displacement by the agent. Alternatively, if the candidate agent is labeled, the presence of the label on the support indicates displacement.

In an alternative embodiment, the candidate agent is added first, with incubation and washing, followed by the competitor. The absence of binding by the competitor may indicate the candidate agent is bound to KSP with a higher affinity. Thus, if the candidate agent is labeled, the presence of the label on the support, coupled with a lack of competitor binding, may indicate the candidate agent is capable of binding to KSP.

It may be of value to identify the binding site of KSP. This can be done in a variety of ways. In one embodiment, once KSP has been identified as binding to the anti-mitotic agent, KSP is fragmented or modified and the assays repeated to identify the necessary components for binding.

Modulation is tested by screening for candidate agents capable of modulating the activity of KSP comprising the steps of combining a candidate agent with KSP, as above, and determining an alteration in the biological activity of KSP. Thus, in this embodiment, the candidate agent should both bind to KSP (although this may not be necessary), and alter its biological or biochemical activity as defined herein. The methods include both in vitro screening methods and in vivo screening of cells for alterations in cell cycle distribution, cell viability, or for the presence, morpohology, activity, distribution, or amount of mitotic spindles, as are generally outlined above.

Alternatively, differential screening may be used to identify drug candidates that bind to the native KSP, but cannot bind to modified KSP.

Positive controls and negative controls may be used in the assays. Preferably all control and test samples are performed in at least triplicate to obtain statistically significant results. Incubation of all samples is for a time sufficient for the binding of the agent to the protein. Following incubation, all samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. As is appreciated by those skilled in the art, those examples prepared by analogous processes may involve variations in synthetic procedure. The example compounds may also be prepared by analogous processes including variations in synthetic procedure within the skill of the art.

### Preparation 1

### 4′-(trifluoromethyl)-4-biphenylamine

A solution of 4-bromoaniline (29 mmol), 4-trifluoromethylphenyl boronic acid (35 mmol), and tetrakis(triphenylphosphine)palladium(0) (1.4 mmol) in 2M aqueous potassium carbonate solution (50 mL) and N,N-dimethylformamide (50 mL) was heated at 100° C. for 17 h. The reaction mixture was cooled, poured into half-saturated aqueous sodium bicarbonate solution (400 mL), and extracted with (3×400 mL) diethyl ether. The combined organic layers were dried over sodium sulfate and concentrated in vacuo. Purification of the residue by flash chromatography (10-30% ethyl acetate/hexanes) provided the title product as a white powder (70%). ESMS [M+H]⁺: 238.2.

### Preparation 2

### [3-fluoro-4′-(trifluoromethyl)-4-biphenylyl]amine

Following the procedure described in Preparation 1 with 4-bromo-2-fluoroaniline provided the title compound. ESMS [M+H]⁺: 256.2.

### Preparation 3

### [4-(2,2,4,4-tetrafluoro-4H-1,3-benzodioxin-6-yl)phenyl]amine

Following the procedure described in Preparation 1 with 4-(4,4,5,5-tetramethyl-1,3,2-dioxaborolan-2-yl)aniline and 6-bromo-2,2,4,4-tetrafluoro-4H-1,3-benzodioxin and purification by Gilson reverse phase HPLC provided the title compound. ESMS [M+H]⁺: 300.2.

### Preparation 4

### 4′-(trifluoromethyl)-3-biphenylamine

Following the procedure described in Preparation 1 with 3-bromoaniline provided the title compound. ESMS [M+H]⁺: 238.2.

### Preparation 5

### 3′-fluoro-4′-(trifluoromethyl)-4-biphenylamine

Following the procedure described in Preparation 1 with 4-bromo-2-fluoro-1-(trifluoromethyl)benzene and 4-(4,4,5,5-tetramethyl-1,3,2-dioxaborolan-2-yl)aniline provided the title compound. ESMS [M+H]⁺: 256.2.

### Preparation 6

### 4′-[(trifluoromethyl)thio]-4-biphenylamine

Following the procedure described in Preparation 1 with 4-bromophenyl trifluoromethyl sulfide and 4-(4,4,5,5-tetramethyl-1,3,2-dioxaborolan-2-yl)aniline provided the title compound. ESMS [M+H]⁺: 270.2.

### Preparation 7

### 4′-[(trifluoromethyl)sulfonyl]-4-biphenylamine

Following the procedure described in Preparation 1 with 4-chlorophenyl trifluoromethyl sulfone and 4-(4,4,5,5-tetramethyl-1,3,2-dioxaborolan-2-yl)aniline provided the title compound. ESMS [M+H]⁺: 302.2.

### Preparation 8

### 4-amino-4′-(trifluoromethyl)-3-biphenylcarbonitrile

Following the procedure described in Preparation 1 with 2-amino-5-bromobenzonitrile provided the title compound. ESMS [M+H]⁺: 263.2.

### Preparation 9

### 3-(methoxy)-4′-(trifluoromethyl)-4-biphenylamine

Following the procedure described in Preparation 1 with 5-chloro-2-nitroanisole provided the intermediate 3-(methoxy)-4-nitro-4′-(trifluoromethyl)biphenyl. ESMS [M+H]⁺: 298.2.

To a solution of 3-(methyoxy)-4-nitro-4′-(trifluoromethyl)biphenyl (1.96 mmol) in acetic acid (10 mL) was added zinc dust (13.7 mmol). The reaction mixture was stirred at room temperature for 2 h and then filtered through Celite and washed with acetic acid (5 mL) and ethanol (5 mL). The filtrate was concentrated in vacuo and the residue was taken up in ethyl acetate (15 mL) and washed with saturated aqueous sodium carbonate solution (10 mL). The organic layer was dried over magnesium sulfate and concentrated in vacuo to give the title compound as an off-white solid. ESMS [M+H]⁺: 268.0.

### Preparation 10

### [3-nitro-4′-(trifluoromethyl)-4-biphenylyl]amine

Following the procedure described in Preparation 1 with 4-bromo-2-nitroaniline provided the title compound. ESMS [M+H]⁺: 283.2.

### Preparation 11

### 4′-methyl-4-biphenylamine

Following the procedure described in Preparation 1 with 4-methylphenyl boronic acid provided the title compound.

### Preparation 12

### 4′-isopropyl-4-biphenylamine

Following the procedure described in Preparation 1 with 4-isopropylphenyl boronic acid provided the title compound. ESMS [M+H]⁺: 212.2.

### Preparation 13

### 4′-t-butyl-4-biphenylamine

Following the procedure described in Preparation 1 with 4-t-butylphenyl boronic acid provided the title compound. ESMS [M+H]⁺: 226.2.

### Preparation 14

### 4-(2,2-difluoro-1,3-benzodioxol-5-yl)aniline

A solution of 5-bromo-2,2-difluoro-1,3-benzodioxole (2.65 mmol), 4-(4,4,5,5-tetramethyl-1,3,2-dioxaborolan-2-yl)aniline (3.97 mmol), tetrakis(triphenylphosphine)palladium(0) (0.08 mmol), and cesium carbonate (7.94 mmol) in N,N-dimethylformamide (8.0 mL) and water (2.0 mL) was heated at 100° C. for 18 h. The reaction mixture was cooled, poured into brine (60 mL), and extracted with ethyl acetate (3×50 mL). The combined organic layers were dried over magnesium sulfate and decolorizing charcoal, filtered through Celite, and concentrated in vacuo. Purification of the residue by Gilson reverse phase HPLC and neutralization of the collected fractions afforded the title product as a white solid (50%). ESMS [M+H]⁺: 250.2.

### Preparation 15

### 2′-chloro-4′-(trifluoromethyl)-4-biphenylamine

Following the procedure described in Preparation 14 with 2-chloro-1-iodo-4-(trifluoromethyl)benzene provided the title compound. ESMS [M+H]⁺: 272.2.

### Preparation 16

### 4-(2,2,3,3-tetrafluoro-2,3-dihydro-1,4-benzodioxin-6-yl)aniline

A solution of 5-bromo-2,2,3,3-tetrafluoro-1,4-benzodioxole (1.04 mmol), 4-(4,4,5,5-tetramethyl-1,3,2-dioxaborolan-2-yl)aniline (1.04 mmol), dichloro[1,1′-bis(diphenylphosphino)ferrocene]palladium(II)•dichloromethane adduct (0.05 mmol), and cesium carbonate (2.08 mmol) in N,N-dimethylformamide (4 mL) and water (1 mL) was heated at 90° C. for 48 h. The reaction was cooled, filtered, and concentrated in vacuo. Purification of the residue by Gilson reverse phase HPLC and neutralization of the collected fractions afforded the title product as a white solid (63%). ESMS [M+H]⁺: 300.2.

### Preparation 17

### 3,3′-difluoro-4′-(trifluoromethyl)-4-biphenylamine

A solution of 2-fluoro-4-iodoaniline (14.8 mmol), bis(pinacolato)diboron (19.2 mmol), potassium acetate (44.3 mmol), and dichloro[1,1′-bis(diphenylphosphino)ferrocene]palladium(II)•dichloromethane adduct (0.45 mmol) in N,N-dimethylformamide (80 mL) was heated at 80° C. for 2 h. The reaction mixture was cooled and was treated with 4-bromo-2-fluorobenzotrifluoride (14.8 mmol), potassium carbonate (73.8 mmol), dichloro[1,1′-bis(diphenylphosphino)ferrocene]palladium(II)•dichloromethane adduct (0.45 mmol), and water (20 mL). The reaction mixture was then heated at 100° C. for 18 h. The reaction mixture was cooled, concentrated in vacuo, dissolved in ethyl acetate (200 mL), filtered, washed with water and brine, dried over sodium sulfate, and concentrated in vacuo. Purification of the residue by flash chromatography (20% ethyl acetate/hexanes) provided the title product as a white solid (69%). ESMS [M+H]⁺: 274.2.

### Preparation 18

### 4-(2,2-difluoro-1,3-benzodioxol-5-yl)-2-fluoroaniline

A solution of 5-bromo-2,2-difluoro-1,3-benzodioxole (1.73 mmol), bis(pinacolato)diboron (1.90 mmol), potassium acetate (5.20 mmol), and dichloro[1,1′-bis(diphenylphosphino)ferrocene]palladium(II)•dichloromethane adduct (0.32 mmol) in N,N-dimethylformamide (8.0 mL) was heated at 80° C. for 2 h. The reaction mixture was cooled and was treated with 2-fluoro-4-iodoaniline (0.86 mmol), dichloro[1,1′-bis(diphenylphosphino)ferrocene]palladium(II)•dichloromethane adduct (0.32 mmol), cesium carbonate (8.65 mmol), and water (2.0 mL). The reaction mixture was then heated at 100° C. for 18 h. The reaction mixture was cooled, poured into brine (60 mL), and extracted with (3×50 mL) ethyl acetate. The combined organic layers were dried over magnesium sulfate and decolorizing charcoal, filtered through Celite, and concentrated in vacuo. Purification of the residue by Gilson reverse phase HPLC and neutralization of the collected fractions afforded the title product as a tan solid (70%). ESMS [M+H]⁺: 267.8.

### Preparation 19

### 2′-chloro-3-fluoro-4′-(trifluoromethyl)-4-biphenylamine

Following the procedure described in Preparation 20 with 2-chloro-1-iodo-4-(trifluoromethyl)benzene provided the title compound. ESMS [M+H]⁺: 290.0.

### Preparation 20

### 4′-[(trifluoromethyl)sulfinyl]-4-biphenylamine

To a stirred solution of 4′-[(trifluoromethyl)thio]-4-biphenylamine (3.71 mmol) in dry dichloromethane (40 mL) at 0° C. under nitrogen was added portionwise m-chloroperoxybenzoic acid (4.82 mmol). The reaction mixture was stirred for 6 h at 0° C. The mixture was filtered, the filtrate was concentrated in vacuo, and the residue was purified by flash chromatography (20-30% ethyl acetate/hexanes) to provide the title product as an orange solid (85%). ESMS [M+H]⁺: 286.2.

### Preparation 21

### 4-(2,2-dimethyl-1,3-benzodioxol-5-yl)aniline

To an ice-cooled solution of 4-bromoveratrole (23.0 mmol) in dichloromethane (10 mL) was added boron tribromide dropwise (34.5 mL of a 1M solution in dichloromethane). The reaction mixture was stirred at reflux for 18 h and then cooled and treated with ice and 6N aqueous sodium hydroxide solution (10 mL). The layers were separated, and the aqueous layer was acidified with 6N aqueous hydrochloric acid solution and extracted with diethyl ether. Concentration of the ether layer provided the crude diol intermediate, which was dissolved in toluene (30 mL) and treated with 2,2-dimethoxypropane (28 mmol) and phosphorus pentoxide (0.023 mmol). The reaction mixture was heated at 90° C. for 4 h. The cooled reaction mixture washed with saturated aqueous sodium carbonate solution and brine and then concentrated in vacuo. Purification of the residue by flash chromatography (5-60% ethyl acetate/hexanes) provided the intermediate 5-bromo-2,2-dimethyl-1,3-benzodioxole in 44% yield. ¹H NMR (400 MHz, CDCl₃): δ 6.93-6.82 (m, 2H), 6.63-6.60 (m, 1H), 1.69 (s, 6H).

A solution of 5-bromo-2,2-dimethyl-1,3-benzodioxole (2.6 mmol), 4-(4,4,5,5-tetramethyl-1,3,2-dioxaborolan-2-yl)aniline (3.1 mmol), and tetrakis(triphenylphosphine)palladium(0) (0.26 mmol) in acetonitrile (2.6 mL) and 2M aqueous sodium carbonate solution (2.6 mL) was irradiated in a Personal Chemistry Emrys Optimizer microwave at 150° C. for 500 s. The reaction mixture was filtered and concentrated in vacuo. The pure title product was isolated upon precipitation using 80:20 diethyl ether/hexanes (31%). ESMS [M+H]⁺: 242.0.

### Preparation 22

### N-methyl-4′-(trifluoromethyl)-4-biphenylamine

To a solution of 4′-(trifluoromethyl)-4-biphenylamine (4.21 mmol) in dichloromethane (30 mL) and pyridine (6.31 mmol) was added 4-nitrophenyl formate (4.21 mmol). The reaction mixture was stirred at room temperature for 18 h, then diluted with ethyl acetate (150 mL) and washed with water (3×100 mL) and brine (1×100 mL). The organic layer was dried over sodium sulfate, filtered, and concentrated in vacuo to provide the crude [4′-(trifluoromethyl)-4-biphenylyl]formamide intermediate. ESMS [M+H]⁺: 266.2.

Crude [4′-(trifluoromethyl)-4-biphenylyl]formamide (2.1 mmol) was dissolved in a solution of 1M lithium aluminum hydride in THF (20 mL). The reaction mixture was stirred at room temperature for 18 h and then quenched with 0.5N aqueous sodium hydroxide solution (20 mL) and extracted with ethyl acetate (3×50 mL). The combined organic layers were dried over sodium sulfate, filtered, and concentrated in vacuo. Recrystallization of the residue with hot isopropyl alcohol provided the title product as a white solid (60%). ESMS [M+H]⁺: 252.2.

### Preparation 23

### 3-bromo-5-fluoro-4′-(trifluoromethyl)-4-biphenylamine

To a solution of 3-fluoro-4′-(trifluoromethyl)-4-biphenylamine (1.9 mmol) in acetic acid (5 mL) was added bromine (2.9 mmol). The reaction mixture was stirred at room temperature for 2 h. The mixture was concentrated in vacuo, dissolved in ethyl acetate (20 mL), and washed with saturated aqueous sodium bicarbonate solution (3×10 mL). The organic layer was dried over sodium sulfate, filtered, and concentrated in vacuo to afford the crude title compound as a brown solid, which was used directly in the next reaction. ESMS [M+H]⁺: 335.2

### Example 1

### Ex 1a N-[4′-(trifluoromethyl)-3-biphenylyl]urea

To a solution of 4′-(trifluoromethyl)-3-biphenylamine (0.21 mmol) in dichloromethane (2.0 mL) was added chlorosulfonylisocyanate (0.25 mmol). The reaction mixture was stirred at room temperature for 3 h, during which a white precipitate formed. The reaction mixture was quenched with water (3.0 mL) and stirred 18 h. The mixture was filtered, washed with hexanes, and the solid dried in vacuo to afford the title compound as a white solid (85%). Alternatively, the product could be purified by Gilson reverse phase HPLC or by flash chromatography (50-90% ethyl acetate/hexanes). ESMS [M+H]⁺: 281.2.

### Ex 1b N-(4′-methyl-4-biphenylyl)urea

Following the procedure described in Example 1a with (4′-methyl-4-biphenylyl)amine provided the title product. ESMS [M+H]⁺: 227.2.

### Ex 1c N-(4′-isopropyl-4-biphenylyl)urea

Following the procedure described in Example 1a with (4′-isopropyl-4-biphenylyl)amine provided the title product. ESMS [M+H]⁺: 255.2.

### Ex 1d N-(4′-t-butyl-4-biphenylyl)urea

Following the procedure described in Example 1a with (4′-t-butyl-4-biphenylyl)amine provided the title product. ESMS [M+H]⁺: 269.2.

### Ex 1e N-{4′-[(trifluoromethyl)thio]-4-biphenyl}urea

Following the procedure described in Example 1a with 4′-[(trifluoromethyl)thio]-4-biphenylamine provided the title product. ESMS [M+H]⁺: 313.0.

### Ex 1f N-[4′-(trifluoromethyl)-4-biphenylyl]urea

Following the procedure described in Example 1a with 4′-(trifluoromethyl)-4-biphenylamine provided the title product. ESMS [M+H]⁺: 281.2.

### Ex 1g N-[3′-fluoro-4′-(trifluoromethyl)-4-biphenylyl]urea

Following the procedure described in Example 1a with 3′-fluoro-4′-(trifluoromethyl)-4-biphenylamine provided the title product. ESMS [M+H]⁺: 299.0.

### Ex 1h N-{4′-[(trifluoromethyl)sulfonyl]-4-biphenylyl}urea

Following the procedure described in Example 1a with 4′-[(trifluoromethyl)sulfonyl]-43-biphenylamine provided the title product. ESMS [M+H]⁺: 345.2.

### Ex 1i N-methyl-N-[4′-(trifluoromethyl)-4-biphenylyl]urea

Following the procedure described in Example 1a with N-methyl-4′-(trifluoromethyl)-4-biphenylamine provided the title product. ESMS [M+H]⁺: 295.2.

### Ex 1j N-[3-bromo-5-fluoro-4′-(trifluoromethyl)-4-biphenylyl]urea

Following the procedure described in Example 1a with 3-bromo-5-fluoro-4′-(trifluoromethyl)-4-biphenylamine provided the title product. ESMS [M+H]⁺: 378.2.

### Ex 1k N-[4-(2,2,3,3-tetrafluoro-2,3-dihydro-1,4-benzodioxin-6-yl)phenyl]urea

Following the procedure described in Example 1a with 4-(2,2,3,3-tetrafluoro-2,3-dihydro-1,4-benzodioxin-6-yl)aniline provided the title product. ESMS [M+H]⁺: 343.2.

### Ex 1l N-[3-fluoro-4′-(trifluoromethyl)-4-biphenylyl]urea

Following the procedure described in Example 1a with [3-fluoro-4′-(trifluoromethyl)-4-biphenylyl]amine provided the title product. ESMS [M+H]⁺: 299.2.

### Ex 1m N-[3-cyano-4′-(trifluoromethyl)-4-biphenylyl]urea

Following the procedure described in Example 1a with 4-amino-4′-(trifluoromethyl)-3-biphenylcarbonitrile provided the title product. ESMS [M+H]⁺: 306.4.

### Ex 1n N-[3-methoxy-4′-(trifluoromethyl)-4-biphenylyl]urea

Following the procedure described in Example 1a with [3-(methoxy)-4′-(trifluoromethyl)-4-biphenylyl]amine provided the title product. ESMS [M+H]⁺: 311.4.

### Ex 1o N-[4-(2,2-difluoro-1,3-benzodioxol-5-yl)phenyl]urea

Following the procedure described in Example 1a with 4-(2,2-difluoro-1,3-benzodioxol-5-yl)aniline provided the title product. ESMS [M+H]⁺: 293.2.

### Ex 1p N-[4-(2,2-difluoro-1,3-benzodioxol-5-yl)-2-fluorophenyl]urea

Following the procedure described in Example 1a with 4-(2,2-difluoro-1,3-benzodioxol-5-yl)-2-fluoroaniline provided the title product. ESMS [M+H]⁺: 311.2.

### Ex 1q N-[2′-chloro-4′-(trifluoromethyl)-4-biphenylyl]urea

Following the procedure described in Example 1a with 2′-chloro-4′-(trifluoromethyl)-4-biphenylamine provided the title product. ESMS [M+H]⁺: 315.0.

### Ex 1r N-[2′-chloro-3-fluoro-4′-(trifluoromethyl)-4-biphenylyl]urea

Following the procedure described in Example 1a with 2′-chloro-3-fluoro-4′-(trifluoromethyl)-4-biphenylamine provided the title product. ESMS [M+H]⁺: 333.2.

### Preparation 24

### N-[3-nitro-4′-(trifluoromethyl)-4-biphenylyl]urea

Following the procedure described in Example 1a with [3-nitro-4′-(trifluoromethyl)-4-biphenylyl]amine provided the title product. ESMS [M+H]⁺: 326.2.

### Example 2

Ex 2a N-[4′-[(trifluoromethyl)sulfinyl]-4-biphenylyl]urea:

To a solution of 4′-[(trifluoromethyl)sulfinyl]-4-biphenylamine (0.35 mmol) in acetic acid (3.0 mL) was added potassium cyanate (1.05 mmol) and water (0.2 mL). After stirring for 18 h at room temperature, the reaction mixture was concentrated in vacuo, dissolved in ethyl acetate (20 mL), and washed with saturated aqueous sodium bicarbonate solution (3×10 mL). The organic layer was dried over sodium sulfate, filtered, and concentrated in vacuo. Purification of the residue by Gilson reverse phase HPLC afforded the title product as a white solid (70%). ESMS [M+H]⁺: 329.2.

### Ex 2b N-[4-(2,2,4,4-tetrafluoro-4H-1,3-benzodioxin-6-yl)phenyl]urea

Following the procedure described in Example 2a with 4-(2,2,4,4-tetrafluoro-4H-1,3-benzodioxin-6-yl)aniline provided the title product. ESMS [M+H]⁺: 343.2.

### Ex 2c N-[4-(2,2-dimethyl-1,3-benzodioxol-5-yl)phenyl]urea

Following the procedure described in Example 2a with 4-(2,2-dimethyl-1,3-benzodioxol-5-yl)aniline provided the title product. ESMS [M+H]⁺: 285.2.

### Ex 2d N-[3,3′-difluoro-4′-(trifluoromethyl)-4-biphenylyl]urea

Following the procedure described in Example 2a with 3,3′-difluoro-4′-(trifluoromethyl)-4-biphenylamine and purification of the residue by flash chromatography (5-10% methanol/dichloromethane) afforded the title product. ESMS [M+H]⁺: 317.2.

### Ex 2e N-[3-hydroxy-4′-(trifluoromethyl)-4-biphenylyl]urea

To a solution of [3-(methyoxy)-4′-(trifluoromethyl)-4-biphenylyl]amine (0.68 mmol) in 1-methyl-2-pyrrolidinone (0.34 mL) was added potassium carbonate (0.034 mmol) followed by thiophenol (0.68 mmol). The reaction mixture was irradiated in a Personal Chemistry Emrys Optimizer microwave at 220° C. for 15 min. The reaction mixture was diluted with ethyl acetate (10 mL) and concentrated in vacuo. Purification of the residue by silica gel chromatography (Isco Combiflash, 20-65% ethyl acetate/hexanes) afforded the intermediate 4-amino-4′-(trifluoromethyl)-3-biphenylol as a brown solid (82%). ESMS [M+H]⁺: 254.2.

Following the procedure described in Example 2a with 4-amino-4′-(trifluoromethyl)-3-biphenylol provided the title product. ESMS [M+H]⁺: 297.4.

### Example 3

### N-[3-amino-4′-(trifluoromethyl)-4-biphenylyl]urea

A solution of N-[3-nitro-4′-(trifluoromethyl)-4-biphenylyl]urea (0.4 mmol) in ethanol (20 mL) was hydrogenated in a Parr shaker at 50 psi hydrogen using 10% palladium on carbon (26 mg). After 2 h, the reaction mixture was filtered through Celite and concentrated in vacuo. Purification by Gilson reverse phase HPLC afforded the title product as a white solid (14%). ESMS [M+H]⁺: 296.2.

### Preparation 25

### {4-[(aminocarbonyl)amino]phenyl}boronic acid

To a solution of 4-(4,4,5,5-tetramethyl-1,3,2-dioxaborolan-2-yl)aniline (1.35 mmol) in dichloromethane (2 mL) was added chlorosulfonyl isocyanate (1.49 mmol). The reaction was stirred at room temperature for 18 h at which point 0.1N aqueous sodium hydroxide solution (1 mL) was added. The mixture was stirred for 2 h and then concentrated in vacuo to remove dichloromethane. After 66 h, a white preciptate formed and was filtered to give the title compound (54%). ESMS [M+H]⁺: 181.2.

### Preparation 26

### 3-(4-iodophenyl)-3-(trifluoromethyl)-3H-diazirine

A solution of 3-(4-iodophenyl)-3-(trifluoromethyl)diaziridine (Topin, A. N., et al. Nucleosides Nucleotides 1998, 17(7), 1163-1176) (1.1 mmol) in anhydrous methanol (12 mL) under nitrogen was cooled to 0° C. and treated with triethylamine (1 mL). Iodine (1.97 mmol) was added in five small portions over 3 min, at which point the resulting red solution was concentrated in vacuo. Brine (10 mL) was added to the residue, and the organics were extracted with diethyl ether (4×10 mL). The combined ethereal extracts were dried over magnesium sulfate and concentrated in vacuo. Purification by silica gel chromatography (Isco Combi-Flash, 0-5% ethyl acetate/hexanes) afforded the title compound as an oil (60%). ¹H NMR (400 MHz, MeOH-d₄) δ 7.85 (d, 2H, J=8.7 Hz), 7.02 (d, 2H, J=8.6 Hz).

### Example 4

### N-[4′-[3-(trifluoromethyl)-3-diaziridinyl]-4-biphenylyl]urea

A solution of 3-(4-iodophenyl)-3-(trifluoromethyl)diaziridine (Topin, A. N., et al. Nucleosides Nucleotides 1998, 17(7), 1163-1176) (0.22 mmol), {4-[(aminocarbonyl)amino]phenyl}boronic acid (0.28 mmol), and tetrakis(triphenylphosphine)palladium(0) (0.01 mmol) in 2M aqueous potassium carbonate solution (1 mL) and N,N-dimethylformamide (1 mL) under nitrogen was stirred at room temperature for 3 days and then at 50° C. for 1 day. The reaction mixture was quenched with 0.1N aqueous sodium hydroxide solution (1 mL) and extracted with ethyl acetate (2×10 mL). The combined organic layers were washed with brine (1 mL), dried over magnesium sulfate, and concentrated in vacuo. Purification of the residue by Gilson reverse phase HPLC afforded the title compound as a white solid (15%). ESMS [M+H]⁺: 323.2.

### Example 5

### N-{4′-[3-(trifluoromethyl)-3H-diazirin-3-yl]-4-biphenylyl}urea

Following the procedure described in Example 1a with 4-(4,4,5,5-tetramethyl-1,3,2-dioxaborolan-2-yl)aniline provided the intermediate N-[4-(4,4,5,5-tetramethyl-1,3,2-dioxaborolan-2-yl)phenyl]urea. ESMS [M+H]⁺: 263.2.

A solution of 3-(4-iodophenyl)-3-(trifluoromethyl)-3H-diazirine (0.18 mmol), N-[4-(4,4,5,5-tetramethyl-1,3,2-dioxaborolan-2-yl)phenyl]urea (0.27 mmol), and tetrakis(triphenylphosphine)palladium(0) (0.01 mmol) in 2M aqueous potassium carbonate solution (1 mL) and N,N-dimethylformamide (1 mL) was stirred at room temperature for 6 days in the dark. The reaction mixture was quenched with 0.1N aqueous sodium hydroxide solution (1 mL) and extracted with ethyl acetate (2×10 mL). The combined organic layers were washed with brine (1 mL), dried over magnesium sulfate, and concentrated in vacuo. Purification of the residue by Gilson reverse phase HPLC afforded the title compound as a white solid (20%). ESMS [M+H]⁺: 321.0.

### Example 6

### N-hydroxy-N′-[4′-(trifluoromethyl)-4-biphenylyl]urea

To a solution of 1-bromo-4-isocyanatobenzene (1.2 mmol) in dichloromethane (3.0 mL) was added hydroxylamine (1.5 mmol). The reaction mixture was stirred at room temperature for 18 h. The formed white precipitate was filtered and washed with cold hexanes to afford the N-(4-bromophenyl)-N-hydroxyurea intermediate as a white solid (85%). ESMS [M+H]⁺: 232.4.

A solution of N-(4-bromophenyl)-N′-hydroxyurea (0.53 mmol), 4-trifluoromethylphenyl boronic acid (0.79 mmol), and tetrakis(triphenylphosphine)palladium(0) (0.026 mmol) in 2M aqueous potassium carbonate solution (1 mL) and 1,4-dioxane (1 mL) was heated at 90° C. for 17 h. The reaction mixture was cooled, poured into half-saturated aqueous sodium bicarbonate solution (10 mL), and extracted with (3×10 mL) diethyl ether. The combined organic layers were dried over sodium sulfate and concentrated in vacuo. Purification of the residue by flash column chromatography (20% ethyl acetate/hexane) afforded the title product as a white solid (55%). ESMS [M+H]⁺: 297.2.

### Preparation 27

### N-(4-bromo-2-fluorophenyl)urea

A solution of 4-bromo-2-fluoroaniline (0.16 mol) in dichloromethane (250 mL) was added dropwise to an ice-cooled solution of chlorosulfonyl isocyanate (0.18 mol) in dichloromethane (750 mL). The reaction mixture was stirred at ambient temperature for 18 h then evaporated under reduced pressure. The resulting residue was treated with ice-cold water (500 mL) then slurried for 6 h. The solids were collected by filtration, rinsed with water, and dried in vacuo. The powder was recrystallized from ethyl acetate/methanol (5:1) to give the title compound as colorless needles (70%). ESMS [M+H]⁺: 233.2.

### Example 7

### Ex 7a N-(3′,4′-dichloro-3-fluoro-4-biphenylyl)urea

A slurry of N-(4-bromo-2-fluorophenyl)urea (0.48 mmol), 3,4-dichlorophenylboronic acid (0.58 mmol), dichloro[1,1′-bis(diphenylphosphino)ferrocene]palladium(II)•dichloromethane adduct (0.01 mmol), and 2M aqueous sodium carbonate solution (1.44 mmol) in acetonitrile (2.0 mL) was irradiated in a Personal Chemistry Emrys Optimizer microwave at 100° C. for 400 s. The reaction mixture was treated with decolorizing charcoal (250 mg) and anhydrous sodium sulfate (250 mg) and filtered through a nylon-membrane syringe filter. Direct purification of the filtrate by Gilson reverse phase HPLC afforded the title compound as a tan solid. ESMS [M+H]⁺: 299.2.

### Ex 7b N-(3-fluoro-4′-Propyl-4-biphenylyl)urea

Following the procedure described in Example 7a with 4-propylphenylboronic acid provided the title product. ESMS [M+H]⁺: 273.2.

### Ex 7c N-[4-(2,3-dihydro-1-benzofuran-5-yl)-2-fluorophenyl]urea

Following the procedure described in Example 7a with 2,3-dihydro-1-benzofuran-5-ylboronic acid at 110° C. provided the title product. ESMS [M+H]⁺: 273.2.

### Ex 7d N-(3-fluoro-3′,4′-dimethyl-4-biphenylyl)urea

Following the procedure described in Example 7a with (3,4-dimethylphenyl)boronic acid at 110° C. provided the title product. ESMS [M+H]⁺: 259.2.

### Ex 7e N-(3-fluoro-4′-isobutyl-4-biphenylyl)urea

Following the procedure described in Example 7a with (4-isobutylphenyl)boronic acid at 110° C. provided the title product. ESMS [M+H]⁺: 287.2.

### Ex 7f N-(3,3′,4′,5′-tetrafluoro-4-biphenylyl)urea

Following the procedure described in Example 7a with (3,4,5-trifluorophenyl)boronic acid at 110° C. provided the title product. ESMS [M+H]⁺: 285.2.

### Ex 7g N-(4′-bromo-3,3′-difluoro-4-biphenylyl)urea

Following the procedure described in Example 7a with (4-bromo-3-fluorophenyl)boronic acid at 110° C. provided the title product. ESMS [M+H]⁺: 327.0.

### Ex 7h N-(4′-ethenyl-3-fluoro-4-biphenylyl)urea

Following the procedure described in Example 7a with (4-ethenylphenyl)boronic acid at 110° C. provided the title product. ESMS [M+H]⁺: 257.2.

### Example 8

### N-[4′-(trifluoromethyl)-4-biphenylyl]thiourea

A solution of 4′-(trifluoromethyl)-4-biphenylamine (0.84 mmol) in water (1 mL) and 1M hydrochloric acid solution (0.84 mL) was treated with ammonium thiocyanate (0.84 mmol). The reaction mixture was heated at 110° C. for 2 h. The homogeneous reaction solution was cooled and poured onto ice (4 g). A white precipitate formed and was collected by filtration. Purification of the residue by Gilson reverse phase HPLC yielded the title product (21%). ESMS [M+H]⁺: 297.

### Example 9

### N-[3,3′-difluoro-4′-(trifluoromethyl)-4-biphenylyl]thiourea

A solution of 3,3′-difluoro-4′-(trifluoromethyl)-4-biphenylamine (1.0 mmol) in tetrahydrofuran (10 mL) and 1N aqueous hydrochloric acid solution (1.0 mL) was treated with ammonium thiocyanate (3.3 mmol). The reaction mixture was heated at reflux for 18 h, at which point additional ammonium thiocyanate (6.6 mmol) was added. After heating at reflux for an additional 3 days, the reaction mixture was cooled to room temperature, concentrated in vacuo, dissolved in ethyl acetate (100 mL), washed with water and brine, dried over magnesium sulfate, and concentrated in vacuo. Purification of the residue by flash chromatography (10% ethyl acetate/hexanes) provided the title compound as an off-white solid (25%). ESMS [M+H]⁺: 333.2.

### Example 10

### [4′-(trifluoromethyl)-4-biphenylyl]formamide

To a solution of 4′-(trifluoromethyl)-4-biphenylamine (4.21 mmol) and pyridine (6.31 mmol) in dichloromethane (30 mL) was added 4-nitrophenyl formate (4.21 mmol). The reaction mixture was stirred at room temperature for 18 h. The reaction mixture was dissolved in ethyl acetate (150 mL) and washed with water (3×100 mL) and brine (1×100 mL). The organic layer was dried over sodium sulfate and was concentrated in vacuo. Purification of the residue by Gilson reverse phase HPLC afforded the title product as a white solid (85%). ESMS [M+H]⁺: 266.2.

### Preparation 28

### (4-bromophenyl)formamide

Following the procedure described in Example 10 with 4-bromoaniline provided the title compound. ESMS [M+H]⁺: 201.2.

### Preparation 29

### (4-bromo-2-fluorophenyl)formamide

Following the procedure described in Example 10 with 4-bromo-2-fluoroaniline provided the title compound. ESMS [M+H]⁺: 219.2.

### Preparation 30

### [4-(4,4,5,5-tetramethyl-1,3,2-dioxaborolan-2-yl)phenyl]formamide

Following the procedure described in Example 10 with [4-(4,4,5,5-tetramethyl-1,3,2-dioxaborolan-2-yl)phenyl]amine provided the title compound. ESMS [M+H]⁺: 248.2.

### Example 11

### Ex 11a [3′-fluoro-4′-(trifluoromethyl)-4-biphenylyl]formamide

A solution of (4-bromophenyl)formamide (0.27 mmol), 2-[3-fluoro-4-(trifluoromethyl)phenyl]-4,4,5,5-tetramethyl-1,3,2-dioxaborolane (0.40 mmol; WO 02/060877), and tetrakis(triphenylphosphine)palladium(0) (0.013 mmol) in 2M aqueous potassium carbonate solution (1 mL) and 1,4-dioxane (1 mL) was heated at 90° C. for 17 h. The reaction mixture was cooled, poured into half-saturated aqueous sodium bicarbonate solution (10 mL), and extracted with (3×10 mL) diethyl ether. The combined organic layers were dried over sodium sulfate and concentrated in vacuo. Purification of the residue by Gilson reverse phase HPLC afforded the title product as a white solid (70%). ESMS [M+H]⁺: 284.2.

### Ex 11b [3,3′-difluoro-4′-(trifluoromethyl)-4-biphenylyl]formamide

Following the procedure described in Example 12a with (4-bromo-2-fluorophenyl)formamide provided the title compound. ESMS [M+H]⁺: 302.2.

### Ex 11c [3-fluoro-4′-(trifluoromethyl)-4-biphenylyl]formamide

Following the procedure described in Example 12a with (4-bromo-2-fluorophenyl)formamide and [4-(trifluoromethyl)phenyl]boronic acid provided the title compound. ESMS [M+H]⁺: 284.2.

### Ex 11d {4′-[(trifluoromethyl)sulfonyl]-4-biphenylyl}formamide

Following the procedure described in Example 12a with 4-chlorophenyl-trifluoromethyl sulfone and [4-(4,4,5,5-tetramethyl-1,3,2-dioxaborolan-2-yl)phenyl]formamide provided the title compound. ESMS [M+H]⁺: 330.2.

### Preparation 31

### 4-nitro-4′-(trifluoromethyl)biphenyl

A solution of 1-bromo-4-nitrobenzene (4.0 mmol), 4-trifluoromethylphenyl boronic acid (5.3 mmol), and tetrakis(triphenylphosphine)palladium(0) (0.12 mmol) in 2M aqueous potassium carbonate solution (10 mL) and N,N-dimethylformamide (10 mL) was heated at 100° C. for 4.7 h. The reaction mixture was cooled, poured into saturated aqueous sodium bicarbonate solution (125 mL), and extracted with (3×100 mL) 1:1 ethyl acetate:hexanes. The combined organic layers were dried over sodium sulfate and concentrated in vacuo. Purification of the residue by flash chromatography (5-25% ethyl acetate/hexanes) provided the title product as an ivory solid (30%). ESMS [M+H]⁺: 268.0.

### Example 12

### hydroxy[4′-(trifluoromethyl)-4-biphenylyl]formamide

To a mixture of 4-nitro-4′-(trifluoromethyl)biphenyl (1.2 mmol) and ammonium chloride (4.6 mmol) in N,N-dimethylformamide (8.0 mL), ethanol (2.0 mL), and water (3.0 mL) was added zinc dust (4.6 mmol). The reaction mixture was stirred at room temperature for 35 min and then was filtered through Celite, rinsing with (2×5 mL) ethanol. The filtrate was poured into brine (50 mL) and water (20 mL), and the organics were extracted with (3×60 mL) chloroform. The combined organic layers were dried over sodium sulfate and were concentrated in vacuo. The crude N-[4′-(trifluoromethyl)-4-biphenylyl]hydroxylamine was used directly in the next step.

To a solution of the crude N-[4′-(trifluoromethyl)-4-biphenylyl]hydroxylamine and diisopropylethylamine (3.4 mmol) in dichloromethane (10 mL) was added p-nitrophenyl formate (1.7 mmol). The reaction mixture was stirred at room temperature for 3 h and then was poured into 1:10 saturated aqueous sodium bicarbonate solution:water (75 mL) and extracted with (2×50 mL) dichloromethane. The combined organic layers were then washed with (7×40 mL) 1:10 saturated aqueous sodium bicarbonate solution:water, or until the aqueous washes were colorless, to remove the p-nitrophenol by-product. The organic layer was dried over sodium sulfate and was concentrated in vacuo. Purification of the residue by flash chromatography (25-55% ethyl acetate/hexanes) provided the title product as a yellow solid (45%, 2 steps). ESMS [M+H]⁺: 282.4.

### Example 13

### [4′-(trifluoromethyl)-4-biphenylyl]thioformamide

To a suspension of phosphorous pentasulfide (0.17 mmol) in acetonitrile (2 mL) was added [4′-(trifluoromethyl)-4-biphenylyl]formamide (0.17 mmol) and sodium sulfate (0.17 mmol). The reaction mixture was stirred at room temperature for 18 h. The reaction mixture was diluted with ethyl acetate (10 mL) and washed with water (3×5 mL). The organic layer was dried over sodium sulfate and was concentrated in vacuo. Purification of the residue by Gilson reverse phase HPLC afforded the title product as a white solid (70%). ESMS [M+H]⁺: 282.2.

### Preparation 32

### (4-bromo-2-fluoro-6-nitrophenyl)formamide

To a solution of (4-bromo-2-fluorophenyl)formamide (2.2 mmol) in concentrated sulfuric acid (10 mL) at 0° C. was added 90% nitric acid (3.3 mmol). The reaction mixture was stirred at 0° C. for 5 min and then at room temperature for 2 h. The reaction mixture was poured into ice water (50 mL) and a precipitate formed which was filtered and dried. The resultant residue was used directly in the next reaction without further purification. ESMS [M+H]⁺: 264.2.

### Preparation 33

### [3-fluoro-5-nitro-4′-(trifluoromethyl)-4-biphenylyl]formamide

A solution of (4-bromo-2-fluoro-6-nitrophenyl)formamide (0.75 mmol), [4-(trifluoromethyl)phenyl]boronic acid (1.13 mmol), and dichloro[1,1′-bis(diphenylphosphino)ferrocene]palladium(II) (0.037 mmol) in 2M aqueous potassium carbonate solution (1 mL) and 1,4-dioxane (1 mL) was irradiated in a Personal Chemistry Emrys Optimizer microwave at 100° C. for 300 s. The reaction mixture was cooled, poured into half-saturated aqueous sodium bicarbonate solution (10 mL), and extracted with (3×10 mL) diethyl ether. The combined organic layers were dried over sodium sulfate and were concentrated in vacuo. Purification of the residue by Gilson reverse phase HPLC afforded the title product as a white solid (75%). ESMS [M+H]⁺: 314.2.

### Example 14

### [3-amino-5-fluoro-4′-(trifluoromethyl)-4-biphenylyl]formamide

A solution of [3-fluoro-5-nitro-4′-(trifluoromethyl)-4-biphenylyl]formamide (0.15 mmol) in methanol (20 mL) was hydrogenated at atmospheric pressure using 10% palladium on carbon (10% w/w). After 2 h at room temperature, the reaction mixture was filtered through Celite and the filtrate was concentrated in vacuo. Purification of the residue by Gilson reverse phase HPLC afforded the title product as a tan solid (85%). ESMS [M+H]⁺: 299.2.

### Example 15

### methyl[4′-(trifluoromethyl)-4-biphenylyl]formamide

To a solution of [4′-(trifluoromethyl)-4-biphenylyl]formamide (0.13 mmol) in dioxane (10 mL) was added sodium hydride (0.19 mmol of a 60% dispersion in mineral oil). The reaction mixture was stirred at room temperature for 15 min. Iodomethane (0.13 mmol) was added and the reaction mixture was stirred for 18 h. The reaction was carefully quenched with water (20 mL) and extracted with ethyl acetate (3×20 mL). The combined organic layers were dried over sodium sulfate and were concentrated in vacuo. Purification of the residue by Gilson reverse phase HPLC afforded the title product as a white solid (85%). ESMS [M+H]⁺: 280.2.

### Example 16

### Monopolar Spindle Formation Following Application of a KSP Inhibitor of the Invention

Human tumor cells SKOV-3 (ovarian) were plated in 96-well plates at densities of 4,000 cells per well, allowed to adhere for 24 hours, and treated with various concentrations of test compounds of the present invention for 24 hours. Cells were fixed in 4% formaldehyde and stained with antitubulin antibodies (subsequently recognized using fluorescently-labeled secondary antibody) and Hoechst dye (which stains DNA).Compounds of this class were found by visual inspection to cause cell cycle arrest in the prometaphase stage of mitosis, although results varied. Where cell cycle arrest was evident, DNA was condensed and spindle formation had initiated, but arrested cells uniformly displayed monopolar spindles, indicating that there was an inhibition of spindle pole body separation. Microinjection of anti-KSP antibodies also causes mitotic arrest with arrested cells displaying monopolar spindles. Although most of the compounds that inhibited KSP activity biochemically did exhibit cell cycle arrest, for some, cell cycle arrest was not detected.

### Example 17

### Inhibition of Cellular Proliferation in Tumor Cell Lines Treated with KSP Inhibitors of the Invention

Cells were plated in 96-well plates at densities from 1000-2500 cells/well of a 96-well plate and allowed to adhere/grow for 24 hours. They were then treated with various concentrations of compounds of the present invention for 48 hours. The time at which compounds are added is considered T₀. A tetrazolium-based assay using the reagent 3-(4,5-dimethylthiazol-2-yl)-5-(3-carboxymethoxyphenyl)-2-(4-sulfophenyl)-2H-tetrazolium (MTS) (U.S. Pat. No. 5,185,450) (see Promega product catalog #G3580, CellTiter 96® AQᵤₑₒᵤₛ One Solution Cell Proliferation Assay) was used to determine the number of viable cells at T₀ and the number of cells remaining after 48 hours compound exposure. The number of cells remaining after 48 hours was compared to the number of viable cells at the time of drug addition, allowing for calculation of growth inhibition.

The growth over 48 hours of cells in control wells that had been treated with vehicle only (0.25% DMSO) is considered 100% growth and the growth of cells in wells with compounds is compared to this. Compounds of this class were found to inhibit cell proliferation in human ovarian tumor cell lines (SKOV-3), although results varied. Although most of the compounds that inhibited KSP activity biochemically did inhibit cell proliferation, for some compounds inhibition was relatively low or not detected. A GI₅₀ was calculated by plotting the concentration of compound in μM vs the percentage of cell growth of cell growth in treated wells. The GI₅₀ calculated for the compounds is the estimated concentration at which growth is inhibited by 50% compared to control, i.e., the concentration at which:
100×[(Treated₄₈−T₀)/(Control₄₈−T₀)]=50.
All concentrations of compounds are tested in duplicate and controls are averaged over 12 wells. A very similar 96-well plate layout and GI₅₀ calculation scheme is used by the National Cancer Institute (see Monks, et al., J. Natl. Cancer Inst. 83:757-766 (1991)). However, the method by which the National Cancer Institute quantitates cell number does not use MTS, but instead employs alternative methods.

Compounds of this class were found to inhibit cell proliferation, although GI₅₀ values varied. GI₅₀ values for the compounds tested ranged from about 100 nM to greater than the highest concentration tested. By this we mean that although most of the compounds that inhibited KSP activity biochemically did inhibit cell proliferation, for some, at the highest concentration tested (generally about 20 μM), cell growth was inhibited less than 50%. Many of the compounds have GI₅₀ values less than 10 μM, and several have GI₅₀ values less than 1 μM. Anti-proliferative compounds that have been successfully applied in the clinic to treatment of cancer (cancer chemotherapeutics) have GI₅₀'s that vary greatly. For example, in A549 cells, paclitaxel GI₅₀ is 4 nM, doxorubicin is 63 nM, 5-fluorouracil is 1 μM, and hydroxyurea is 500 μM (data provided by National Cancer Institute, Developmental Therapeutic Program, http://dtp.nci.nih.gov/). Therefore, compounds that inhibit cellular proliferation at virtually any concentration may be useful. However, preferably, compounds will have GI₅₀ values of less than 1 mM. More preferably, compounds will have GI₅₀ values of less than 20 μM. Even more preferably, compounds will have GI₅₀ values of less than 10 μM. Further reduction in GI₅₀ values may also be desirable, including compounds with GI₅₀ values of less than 1 μM. Some of the compounds of the invention inhibit cell proliferation with GI₅₀ values below 200 nM.

### Example 18

### Calculation of IC₅₀

Measurement of a compound's IC₅₀ for KSP activity uses an ATPase assay. The following solutions are used: Solution 1 consists of 2 mM phosphoenolpyruvate potassium salt (Sigma P-7127), 0.03-1 mM ATP (Sigma A-3377), 1 mM DTT (Sigma D-9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT400301), and 1 mM EGTA (Sigma E3889). Solution 2 consists of 0.6 mM NADH (Sigma N8129), 0.2 mg/mL BSA (Sigma A7906), pyruvate kinase 7 U/mL, L-lactate dehydrogenase 10 U/mL (Sigma P0294), 50-100 nM KSP motor domain, 200 μg/mL microtubules, 1 mM DTT (Sigma D9779), 10 μM paclitaxel (Sigma T-7402), 250 ppm antifoam 289 (Sigma A-8436), 25 mM Pipes/KOH pH 6.8 (Sigma P6757), 2 mM MgCl₂ (VWR JT4003-01), and 1 mM EGTA (Sigma E3889). Serial dilutions (8-12 two-fold dilutions) of the compound are made in a 96-well microtiter plate (Corning Costar 3695) using Solution 1. Following serial dilution each well has 501 μl of Solution 1. The reaction is started by adding 50 μl of solution 2 to each well. This may be done with a multichannel pipettor either manually or with automated liquid handling devices. The microtiter plate is then transferred to a microplate absorbance reader and multiple absorbance readings at 340 nm are taken for each well in a kinetic mode. The observed rate of change, which is proportional to the ATPase rate, is then plotted as a function of the compound concentration. For a standard IC₅₀ determination the data acquired is fit by the following four parameter equation using a nonlinear fitting program (e.g., Grafit 4):

$$y = \frac{Range}{1 + {( \frac{x}{{IC}_{50}} )}^{s}} + Background$$

where y is the observed rate and x the compound concentration.

The compounds of the Examples exhibited a KSP IC₅₀ of 20 μM or less using an ATP concentration of 0.015 mM.

Certain intermediate compounds useful for preparing the compounds of formula I also possess anti-mitotic activity as described above (e.g., as shown by IC₅₀). Such intermediate compounds and their use in the pharmaceutical compositions and the methods described herein also form part of the present invention. Examples of intermediate compounds which possess anti-mitotic activity are 4′-(trifluoromethyl)-4-biphenylamine, [3-fluoro-4′-(trifluoromethyl)-4-biphenylyl]amine, 4′-(trifluoromethyl)-3-biphenylamine, [4-(2,2,4,4-tetrafluoro-4H-1,3-benzodioxin-6-yl)phenyl]amine, 3′-fluoro-4′-(trifluoromethyl)-4-biphenylamine, 4′-[(trifluoromethyl)thio]-4-biphenylamine, 4′[(trifluoromethyl)sulfonyl]-4-biphenylamine, 4-amino-4′-(trifluoromethyl)-3-biphenylcarbonitrile, 4′-isopropyl-4-biphenylamine, 4′-t-butyl-4-biphenylamine, 4-(2,2,3,3-tetrafluoro-2,3-dihydro-1,4-benzodioxin-6-yl)aniline, 3-bromo-5-fluoro-4′-(trifluoromethyl)-4-biphenylamine, 4-(2,2-difluoro-1,3-benzodioxol-5-yl)-2-fluoroaniline, 3,3′-difluoro-4′-(trifluoromethyl)-4-biphenylamine, and 4-(2,2-difluoro-1,3-benzodioxol-5-yl)aniline.

All publications, including but not limited to patents and patent applications, cited in this specification are herein incorporated by reference as if each individual publication were specifically and individually indicated to be incorporated by reference herein as though fully set forth.

It is to be understood that the present invention covers all combinations of particular and preferred groups described herein above.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

We claim:

1. A compound according to formula I 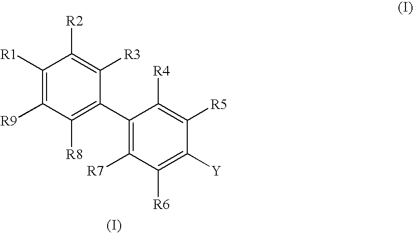
wherein:
R1 is selected from the group consisting of: NR10C(X)Z, H, halo, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and
wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;
X is O or S;
Z is H or NHR11;
R2 is selected from the group consisting of: NR10C(X) Z, H, halo, CN, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and
wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;
provided that one and only one of R1 and R2 is NR10C(X)Z;
R3, R4, R7, and R8, are each independently selected from the group consisting of: H, halo, OH, CN, NO₂, NR12R13, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and
wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;
R5, R6, and R9 are each independently selected from the group consisting of: H, halo, CN, NO₂, NR12R13, OR14, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₃-C₆ cycloalkyl, and heterocycloalkyl, and
wherein said C₃-C₆ cycloalkyl, and heterocycloalkyl are optionally substituted with one or more substituent selected from the group consisting of: ORa, CN, NRbRc, C(O)ORa, C(O)NRbRc, N(Rb)C(O)Re, SO₂NRbRe, N(Rb)SO₂Re, C₁-C₃ alkyl and C₁-C₃ haloalkyl;
R10 is selected from the group consisting of: H, OH, cyclopropyl, and C₁-C₃ alkyl;
R11 is selected from the group consisting of: H, ORf, optionally substituted C₁-C₅ alkyl, optionally substituted C₁-C₅ haloalkyl, optionally substituted C₃-C₆ cycloalkyl,
wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl, and
wherein said C₃-C₆ cycloalkyl is optionally substituted with one or more substituent selected from the group consisting of: ORa, NRbRc, C(O)ORa, C(O)NRbRc, N(Rb)C(O)Re, SO₂NRbRe, N(Rb)SO₂Re, C₁-C₃ alkyl and C₁-C₃ haloalkyl;
R12 is selected from the group consisting of H, C₁-C₃ alkyl, and cyclopropyl;
R13 is selected from the group consisting of: H, ORf, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, C₃-C₆ cycloalkyl, and heterocycloalkyl, and
wherein said C₃-C₆ cycloalkyl and heterocycloalkyl are optionally substituted with one or more substituent selected from the group consisting of: ORa, NRbRc, C(O)ORa, C(O)NRbRc, N(Rb)C(O)Re, SO₂NRbRe, —N(Rb)SO₂Re, C₁-C₃ alkyl and C₁-C₃ haloalkyl;
R14 is selected from the group consisting of: H, optionally substituted C₁-C₅ alkyl, optionally substituted C₁-C₅ haloalkyl, and optionally substituted C₃-C₆ cycloalkyl,
wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl, and
wherein said C₃-C₆ cycloalkyl is optionally substituted with one or more substituent selected from the group consisting of: ORa, NRbRc, C(O)ORa, C(O)NRbRc, N(Rb)C(O)Re, SO₂NRbRe, —N(Rb)SO₂Re, C₁-C₃ alkyl and C₁-C₃ haloalkyl;
when Z is NHR11Y is selected from the group consisting of: halo, OCF₃, S(O)ₙCF₃, optionally substituted C₁-C₅ alkyl, optionally substituted C₁-C₅ haloalkyl, and C(R15)(R16)(CF₃),
wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl,
when Z is H Y is selected from the group consisting of: OCF₃, S(O)ₙCF₃, optionally substituted C₁-C₄ alkyl, optionally substituted C₁-C₅ haloalkyl, and C(R15)(R16)(CF₃),
wherein said C₁-C₄ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl,
or when Z is H or NHR11 Y and either R5 or R6 taken together with the carbon atoms to which they are attached form a ring having from 5 to 7 member atoms wherein said ring optionally contains 1 or 2 heteroatoms as member atoms, said ring is saturated or unsaturated, and said ring is optionally substituted with one or more substituent selected from the group consisting of: halo, ORa, CN, NR12R13, optionally substituted C₁-C₅ alkyl, and optionally substituted C₁-C₅ haloalkyl,
wherein said C₁-C₅ alkyl and C₁-C₅ haloalkyl are optionally substituted with one or more substituent selected from the group consisting of: ORa, NRbRc, C(O)ORa, C(O)NRbRc, N(Rb)C(O)Re, SO₂NRbRe, N(Rb)SO₂Re, and C₃-C₆ cycloalkyl;
R15 and R16 taken together with the carbon to which they are attached form a ring having from 3 to 6 member atoms wherein said ring optionally contains from 1 to 3 heteroatoms as member atoms, said ring is saturated or unsaturated, and said ring is optionally substituted with one or more substituent selected from the group consisting of: halo, —ORa, —CN, optionally substituted C₁-C₅ alkyl, and optionally substituted C₁-C₅ haloalkyl;
n is 0, 1, or 2;
Ra is selected from the group consisting of: H, optionally substituted C₁-C₅ alkyl, optionally substituted C₁-C₅ haloalkyl, and optionally substituted C₃-C₆ cycloalkyl,
wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and
wherein said C₃-C₆ cycloalkyl is optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;
Rb is selected from the group consisting of: H, C₁-C₃ alkyl, and cyclopropyl;
Rc is selected from the group consisting of: H, —ORd, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and
wherein said C₃-C₆ cycloalkyl and heterocycloalkyl are optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;
Rd is selected from the group consisting of: H and C₁-C₃ alkyl;
Re is selected from the group consisting of: H, optionally substituted C₁-C₅ alkyl, optionally substituted C₁-C₅ haloalkyl, optionally substituted C₃-C₆ cycloalkyl, and optionally substituted heterocycloalkyl,
wherein said C₁-C₅ alkyl and C₁-C₅ haloalkyl are optionally substituted with one or more substituent selected from the group consisting of: —OH, —NRdRd, —C(O)OH, C(O)NRdRd, N(Rd)C(O)Rd, SO2NRdRd, and N(Rd)SO2Rd, and
wherein said C₃-C₆ cycloalkyl and heterocycloalkyl are optionally substituted with one or more substituent selected from the group consisting of: OH, NRdRd, C(O)OH, C(O)NRdRd, N(Rd)C(O)Rd, SO2NRdRd, N(Rd)SO2Rd, C₁-C₃ alkyl and C₁-C₃ haloalkyl;
Rf is selected from the group consisting of: H, optionally substituted C₁-C₅ alkyl, optionally substituted C₁-C₅ haloalkyl, and optionally substituted C₃-C₆ cycloalkyl,
wherein said C₁-C₅ alkyl and C₁-C₅ haloalkyl are optionally substituted with one or more substituent selected from the group consisting of: ORd, NRdRd, C(O)OH, C(O)NRdRd, N(Rd)C(O)Rd, SO₂NRdRd, N(Rd)SO₂Rd, and C₃-C₆ cycloalkyl, and
wherein said C₃-C₆ cycloalkyl is optionally substituted with one or more substituent selected from the group consisting of: ORd, NRdRd, C(O)OH, C(O)NRdRd, N(Rd)C(O)Rd, SO₂NRdRd, N(Rd)SO₂Rd, C₁-C₃ alkyl and C₁-C₃ haloalkyl;
or a pharmaceutically acceptable salt thereof.

2. A compound or salt according to claim 1 wherein R1 is NR10C(X)Z.

3. A compound or salt according to claim 1 or 2 wherein R10 is H, OH, or C₁-C₃ alkyl.

4. A compound or salt according to claim 3 wherein R10 is H.

5. A compound or salt according to any of the preceding claims wherein R3, R4, R7, and R8 are each independently selected from the group consisting of H and halo.

6. A compound or salt according to claim 5 wherein R3, R4, R7, and R8 are each H.

7. A compound or salt according to any of the preceding claims wherein Y is selected from the group consisting of: OCF₃, SO₂CF₃, optionally substituted C₁-C₅ alkyl, CF₃, bromo, and C(R15)(R16)(CF₃), provided that when Z is H, Y is not bromo; or Y and either R5 or R6 taken together with the carbon atoms to which they are attached form a ring having from 5 or 6 member atoms wherein said ring optionally contains 1 or 2 heteroatoms as member atoms, said ring is saturated or unsaturated, and said ring is optionally substituted with one or more substituent selected from the group consisting of: halo, ORa, CN, NR12R13, optionally substituted C₁-C₅ alkyl, and optionally substituted C₁-C₅ haloalkyl.

8. A compound or salt according to claim 7 wherein Y is selected from the group consisting of: OCF₃, SO₂CF₃, CF₃, bromo, i-butyl, i-propyl, and t-butyl, provided that when Z is H, Y is not bromo; or Y and either R5 or R6 taken together with the carbon atoms to which they are attached form an optionally substituted ring wherein said ring contains 1 to 2 atoms selected from oxygen and sulfur and is substituted with one or more halo, C₁-C₃ alkyl, and/or halo-C₁-C₃ alkyl groups.

9. A compound or salt according to claim 7 wherein Y is selected from the group consisting of: OCF₃, SO₂CF₃, CF₃, i-propyl, and t-butyl; or Y and either R5 or R6 taken together with the carbon atoms to which they are attached form a substituted ring wherein said ring contains 1 to 2 atoms selected from oxygen and sulfur and is substituted with one or more fluoro, methyl, and/or trifluoromethyl groups.

10. A compound or salt according to any of claims 1-9 wherein Z is NHR11.

11. A compound or salt according to claim 10 wherein R11 is H or OH.

12. A compound or salt according to claim 10 wherein R11 is H.

13. A compound or salt according to any of claims 1-9 wherein Z is H.

14. A compound selected from the group consisting of:
N-[4′-(trifluoromethyl)-3-biphenylyl]urea;
N-(4′-methyl-4-biphenylyl)urea;
N-[4′-(isopropyl)-4-biphenylyl]urea;
N-[4′-(t-butyl)-4-biphenylyl]urea;
N-{4′-[(trifluoromethyl)thio]-4-biphenyl}urea;
N-[4′-(trifluoromethyl)-4-biphenylyl]urea;
N-[3′-fluoro-4′-(trifluoromethyl)-4-biphenylyl]urea;
N-{4′-[(trifluoromethyl)sulfonyl]-4-biphenylyl}urea;
N-methyl-N-[4′-(trifluoromethyl)-4-biphenylyl]urea;
N-[3-bromo-5-fluoro-4′-(trifluoromethyl)-4-biphenylyl]urea;
N-[4-(2,2,3,3-tetrafluoro-2,3-dihydro-1,4-benzodioxin-6-yl)phenyl]urea;
N-[3-fluoro-4′-(trifluoromethyl)-4-biphenylyl]urea;
N-[3-cyano-4′-(trifluoromethyl)-4-biphenylyl]urea;
N-[3-methoxy-4′-(trifluoromethyl)-4-biphenylyl]urea;
N-[4-(2,2-difluoro-1,3-benzodioxol-5-yl)phenyl]urea;
N-[4-(2,2-difluoro-1,3-benzodioxol-5-yl)-2-fluorophenyl]urea;
N-[2′-chloro-4′-(trifluoromethyl)-4-biphenylyl]urea;
N-[2′-chloro-3-fluoro-4′-(trifluoromethyl)-4-biphenylyl]urea;
N-{4′-[(trifluoromethyl)sulfinyl]-4-biphenylyl}urea;
N-[4-(2,2,4,4-tetrafluoro-4H-1,3-benzodioxin-6-yl)phenyl]urea;
N-[4-(2,2-dimethyl-1,3-benzodioxol-5-yl)phenyl]urea;
N-[3,3′-difluoro-4′-(trifluoromethyl)-4-biphenylyl]urea;
N-[3-hydroxy-4′-(trifluoromethyl)-4-biphenylyl]urea;
N-[3-amino-4′-(trifluoromethyl)-4-biphenylyl]urea;
N-{4′-[3-(trifluoromethyl)-3-diaziridinyl]-4-biphenylyl}urea;
N-{4′-[3-(trifluoromethyl)-3H-diazirin-3-yl]-4-biphenylyl}urea;
N-hydroxy-N′-[4′-(trifluoromethyl)-4-biphenylyl]urea;
N-(3′,4′-dichloro-3-fluoro-4-biphenylyl)urea;
N-(3-fluoro-4′-propyl-4-biphenylyl)urea;
N-[4-(2,3-dihydro-1-benzofuran-5-yl)-2-fluorophenyl]urea;
N-(3-fluoro-3′,4′-dimethyl-4-biphenylyl)urea;
N-(3-fluoro-4′-isobutyl-4-biphenylyl)urea;
N-(3,3′,4′,5′-tetrafluoro-4-biphenylyl)urea;
N-(4′-bromo-3,3′-difluoro-4-biphenylyl)urea;
N-(4′-ethenyl-3-fluoro-4-biphenylyl)urea;
N-[4′-(trifluoromethyl)-4-biphenylyl]thiourea;
N-[3,3′-difluoro-4′-(trifluoromethyl)-4-biphenylyl]thiourea;
[4′-(trifluoromethyl)-4-biphenylyl]formamide;
[3′-fluoro-4′-(trifluoromethyl)-4-biphenylyl]formamide;
[3,3′-difluoro-4′-(trifluoromethyl)-4-biphenylyl]formamide;
[3-fluoro-4′-(trifluoromethyl)-4-biphenylyl]formamide;
{4′-[(trifluoromethyl)sulfonyl]-4-biphenylyl}formamide;
hydroxy[4′-(trifluoromethyl)-4-biphenylyl]formamide;
[4′-(trifluoromethyl)-4-biphenylyl]thioformamide;
[3-amino-5-fluoro-4′-(trifluoromethyl)-4-biphenylyl]formamide;
methyl[4′-(trifluoromethyl)-4-biphenylyl]formamide;
and pharmaceutically acceptable salts thereof.

15. A composition comprising a compound or salt according to any of claims 1-14 and pharmaceutically acceptable excipient.

16. A method of modulating or inhibiting KSP activity which comprises contacting said kinesin with an effective amount of a compound or salt according to any of claims 1-14.

17. A method for the treatment of a disease of proliferating cells comprising administering to a patient in need thereof a compound or salt according to any of claims 1-14.

18. A method according to claim 17 wherein the disease is selected from the group consisting of: cancer, hyperplasias, restenosis, cardiac hypertrophy, immune disorders, fungal disorders and inflammation.

19. Use of a compound according to any of claims 1 to 14 in the manufacture of a medicament for use in the treatment of a disease of proliferating cells.

20. A method of preparing a compound of formula (I) comprising either:
a) reacting a compound of the formula (II) 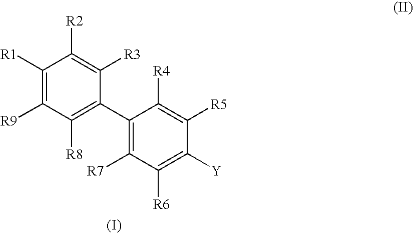
with either an isocyanate, a formate, or ammonium thiocyanate;
wherein one and only one of R1 and R2 are NH₂ and R3-R9 and Y are as defined in claim 1, and wherein the compound of formula (II) is optionally prepared by either
(i) reacting a compound of formula (a): 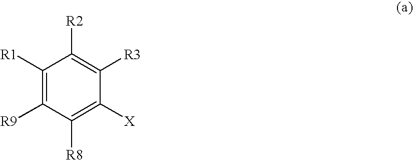
with a compound of formula (b): 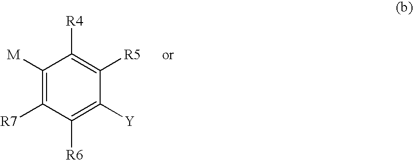
(ii) reacting a compound of formula (c): 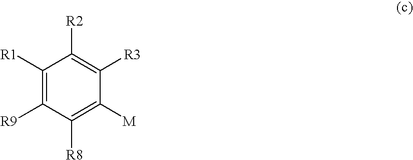
with a compound of formula (d): 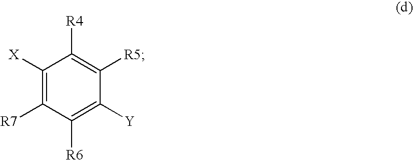
wherein one and only one of R1 and R2 are NH₂, R3-R9 and Y are as defined in claim 1, X is halo, and M is functionalized boron, magnesium, or tin; or
b) reacting a compound of formula (III) 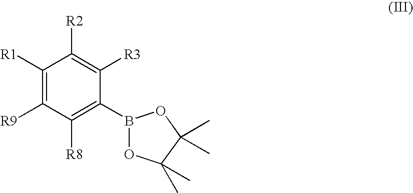
with a compound of formula (IV) 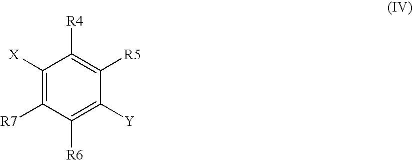
wherein R1-R9 and Y are as defined in claim 1 and X is halo.
